SEQUENCE LISTING (1 sequence)

SEQ ID NO: 1 - DNA, 24 bp, Artificial Sequence
  Synthetic construct
tcatcattttgtcattttgtcatt

(19) United States
(12) Patent Application Publication
(10) Pub. No.: US 2021/0040447 A1
(43) Pub. Date: Feb. 11, 2021

(54) IMMUNE CELL ACTIVATION

(71) Applicant: Claudia Zylberberg, Delray Beach, FL (US)

(72) Inventor: Claudia Zylberberg, Delray Beach, FL (US)

(21) Appl. No.: 16/975,973

(22) PCT Filed: Feb. 26, 2019
(86) PCT No.: PCT/US19/19626

§ 371 Date: Aug. 26, 2020

Related U.S. Application Data
(60) Provisional application No. 62/635,017, filed on Feb. 26, 2018.
(60) Provisional application No. 62/779,844, filed on Dec. 14, 2018.

(51) Int. Cl. C12N 5/0781

(52) U.S. Cl. CPC C12N 5/0635; C12N 2500/40; C12N 2310/315; A61K 45/06; C12N 2501/70; C12N 2501/415; C12N 2501/30; C12N 2501/11

(57) ABSTRACT

The invention refers to a method for preparing novel and powerful regulatory B cells (Breg-nov) by contacting cells obtained from the immune system with phosphorothioate oligonucleotide. Methods of suppressing-autoimmunity or suppressing acute or chronic inflammation or repairing a damaged organ or tissue in mammals, including humans, by administering to the mammals in need, Breg-nov cells obtained as herein described.

### CROSS REFERENCE TO RELATED APPLICATIONS

This Application claims the benefit of U.S. Provisional Application 62/635,017 filed on Feb. 26, 2018 and U.S. Provisional Application 62/779,844 filed on Dec. 14, 2018. The entire contents of these applications are incorporated herein by reference in their entirety.

### BACKGROUND

It is well known, that B cells have a central role in the immune response. B cells act as antigen presenting cells, as activators of other immune active cells by secretion of pro-inflammatory cytokines or by cell to cell contact and by differentiation to antibody producing or memory B cells. In addition, it is now recognize that B cells are also implicate in resolution of the inflammatory phase of the immune response (1). B cells specialized in this anti-inflammatory phase are generically named regulatory B cells (Breg). Bregs are not at all a phenotypically homogeneous group of cells but a group of B cells having in common their capacity to suppress inflammation and autoimmunity and to promote organ and tissue repair by mechanisms determined by stimulus of the milieu (1). These stimuli are probably associated to cell components released by damaged cells or infection agents (2). However, identification of such cell components for B cell induction is largely lacking.

### SUMMARY

Embodiments of the invention are directed to compositions and methods for preparing novel and powerful regulatory B cells (Breg-nov) which produce one or more proteins involved in immune regulatory and/or tissue reparatory process. In certain embodiments these proteins comprise: Neudecin, Pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2, Prostaglandin E2 synthase and combinations thereof. In certain embodiments, a method comprises contacting one or more CD19⁺ B cells, ex-vivo, with the phosphorothioate oligonucleotide. IMT504, having the sequence TCATCATTTTGTCATTTTGTCATT (SEQ ID NO: 1), for at least 20 hours under appropriate conditions. The invention also refers to a method of suppressing-autoimmunity or suppressing acute or chronic inflammation or repairing a damaged organ or tissue in mammals, including humans, by administering, through a convenient parenteral route, to the mammal in need, Breg-nov cells obtained by the inventive method. The starting cells for the procedure are CD19⁺ B cells extracted from say mammal.

In certain embodiments, a method of producing a regulatory B cell comprises: contacting one or more B cells, with a phosphorothioate oligonucleotide having at least a 50% sequence identity to SEQ ID NO: 1, wherein the B cells are CD19⁺ cells. In certain embodiments, the phosphorothioate oligonucleotide has a sequence of at least 70% to SEQ ID NO: 1. In certain embodiments, the CD19⁺ B cells produce one or more proteins associated with immune regulatory and/or tissue reparatory processes. In certain embodiments, the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2. or Prostaglandin E2 synthase. In certain embodiments, the B cells are contacted with the phosphorothioate oligonucleotide for at least about 30 minutes.

In certain embodiments, a method of suppressing an autoimmune response in a subject, comprises obtaining B cells; culturing and contacting the B cells with a phosphorothioate oligonucleotide having at least a 50% sequence identity to SEQ ID NO: 1, administering the B cells to the subject, thereby suppressing the autoimmune response in the subject. In certain embodiments, the phosphorothioate oligonucleotide has a sequence of at least 70% to SEQ ID NO: 1. In certain embodiments, the CD19⁺ B cells produce one or more proteins associated with immune regulatory and/or tissue reparatory processes. In certain embodiments, the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 or Prostaglandin E2 synthase. In certain embodiments, the B cells are cultured ex vivo. In certain embodiments, the B cells are autologous, haplotype matched, cell-lines, stem cells or combinations thereof. In certain embodiments, the method further comprises administering one or more immunosuppressive agents and/or a phosphorothioate oligonucleotide having at least a 50% sequence identity to SEQ ID NO: 1.

In certain embodiments, method of suppressing acute or chronic inflammation or repairing a damaged organ or tissue in mammals comprises obtaining B cells; culturing and contacting the B cells with a phosphorothioate oligonucleotide having at least a 50% sequence identity to SEQ ID NO: 1, administering the B cells to the subject, thereby suppressing the autoimmune response in the subject. In certain embodiments, the phosphorothioate oligonucleotide has a sequence of at least 70% to SEQ ID NO: 1. In certain embodiments, the CD19⁺ B cells produce one or more proteins associated with immune regulatory and/or tissue reparatory processes. In certain embodiments, the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein. Mucin 1, Integrin Subunit Alpha 2 or Prostaglandin E2 synthase. In certain embodiments, the B cells are cultured ex vivo. In certain embodiments, the B cells are autologous, haplotype matched, cell-lines, stem cells or combinations thereof. In certain embodiments, the methods further comprises administering one or more anti-inflammatory agents, other therapeutics and/or a phosphorothioate oligonucleotide having at least a 50% sequence identity to SEQ ID NO: 1.

In certain embodiments, a composition comprises a therapeutically effective amount of a phosphorothioate oligonucleotide having at least a 50% sequence identity to SEQ ID NO: 1 (IMT504), one or more anti-inflammatory agents, other therapeutics, immunosuppressive agents, chemotherapeutic agents or combinations thereof.

In certain embodiments, a method of treating cancer comprises obtaining B cells; culturing and contacting the B cells with a phosphorothioate oligonucleotide having at least a 50% sequence identity to SEQ ID NO: 1, administering the B cells to the subject, thereby treating cancer. In certain embodiments, the method further comprises administering one or more chemotherapeutic agents and/or a phosphorothioate oligonucleotide having at least a 50% sequence identity to SEQ ID NO: 1.

In certain embodiments, a method of regulating an immune response, comprises contacting one or more cells, with a phosphorothioate oligonucleotide having at least a 50% sequence identity to SEQ ID NO: 1. In certain embodiments, the phosphorothioate oligonucleotide has a sequence of at least 70% to SEQ ID NO: 1. In certain embodiments, the one or more cells comprise immune cells. In certain embodiments, the immune cells comprise: B cells, T cells, antigen presenting cells, chimeric antigen receptor-T cells (CAR-T) or combinations thereof. In certain embodiments, the cells are autologous cells, comprising: autologous, allogeneic, haplotype matched, haplotype mismatched, haplo-identical, xenogeneic, cell lines or combinations thereof.

In certain embodiments, a method of treating cancer comprises obtaining immune cells; culturing and contacting the immune cells with a composition comprising a phosphorothioate oligonucleotide having at least a 50% sequence identity to SEQ ID NO: 1 and/or a tumor antigen, and administering the immune cells to the subject, thereby treating cancer. In certain embodiments, the method further comprises administering one or more chemotherapeutic agents and/or a phosphorothioate oligonucleotide having at least a 50% sequence identity to SEQ ID NO: 1. In certain embodiments, the immune cells comprise: B cells, T cells, antigen presenting cells, chimeric antigen receptor-T cells (CAR-T) or combinations thereof. In certain embodiments, the cells are autologous cells, comprising: autologous, allogeneic, haplotype matched, haplotype mismatched, haplo-identical, xenogeneic, cell lines or combinations thereof.

In certain embodiments, a method of producing regulatory B (Breg) cells, comprises contacting B cells ex vivo with a single stranded immunomodulatory oligonucleotide IMT504 with the TCATCATTTTGTCATTTTGTCATT (SEQ ID NO: 1) sequence for about 48 hours wherein the Breg cells produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase.

In certain embodiments, a method for preparing immunomodulatory extracellular vesicles, comprises contacting B-cells ex vivo with an immunomodulatory oligonucleotide having a sequence as set forth in SEQ ID NO: 1 for about 48 hours, and recovering the extracellular vesicles from the cell culture supernatant.

In certain embodiments, a method of producing the cytokine interleukin-35 (IL-35), comprises contacting B cells ex vivo with an immunomodulatory oligonucleotide comprising SEQ ID NO: 1 (IMT504) for about 48 hours, and recovering the IL-35 from the cell culture supernatant.

In certain embodiments, the B cells are primary B cells. In certain embodiments, B cells are cell-line B cells.

In certain embodiments, a method for differentiating cells to an anti-inflammatory and/or pro-reparatory tissue/organ stage or for proliferating cells, in vitro or in vivo, comprises contacting the Breg cells with the cells. In certain embodiments, the cells are monocytes. In certain embodiments, the cells are cells In certain embodiments, the cells are stem cells.

In certain embodiments, a method for autoimmunity treatment in a mammal, comprises administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase.

In certain embodiments, a method for inflammatory disease treatment in a mammal, comprises administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase. In certain embodiments, the inflammatory disease is a chronic inflammatory disease.

In certain embodiments, a method for graft versus host disease prevention or treatment in a mammal, comprises administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase.

In certain embodiments, a method for autoimmunity treatment in a mammal, comprises administering to the mammal extracellular vesicles.

In certain embodiments, a method for inflammatory disease treatment in a mammal, which comprises administering to the mammal exosomes. In certain embodiments, the inflammatory disease is a chronic inflammatory disease.

In certain embodiments, a method for graft versus host disease prevention or treatment in a mammal, which comprises administering to the mammal extracellular vesicles.

Other embodiments are described infra.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3,75, 4.1, and the like) and any range within that range.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term “autoimmunity,” as used herein, refers to the failure of an organism to recognize its own constituent parts as self, resulting in an immune response against the organism's own cells and tissues. “Autoimmune disease” refers to any diseases caused by autoimmunity. Examples of autoimmune diseases are: rheumatoid arthritis, Crohn's disease, inflammatory bowel disease, psoriasis, type 1 diabetes, Myocarditis, Lupus nephritis, Alopecia Areata, Erythema nodosum, Vitiligo, Graves' disease, Ulcerative colitis, Thrombocytopenia, Systemic Lupus Erythematosus, Dermatomyositis, Myasthenia gravis, Guillain-Barré, syndrome, Autoimmune uveitis and Vasculitis. There are more than 100 autoimmune diseases listed by the American Autoimmune Related Diseases Association (aarda.org/diseaselist/).

As used herein, the term “cancer therapy” refers to a therapy useful in treating cancer. Examples of anti-cancer therapeutic agents include, but are not limited to, antibacterial agents as described herein as well as, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAN/43-9006, Bayer Labs.), and Gefitinib (IRESSA™, Astrazeneca), AG-1478, AG-1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYC™ doxorubicin (including morpholino-doxorubicin, cyanomorpholino-doxorubicin, 2-pyrroline-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamide; thiotepa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

As used herein, the term “chemokine” refers to soluble factors cytokines) that have the ability to selectively induce chemotaxis and activation of leukocytes. They also trigger processes of angiogenesis, inflammation, wound healing, and tumorigenesis. Examples of chemokines include IL-8, a human homolog of murine keratinocyte chemoattractant (KC).

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECANT™ topoisomerase 1 inhibitor; ABARELIX™ rmRH; (x) anti-angiogenic agents such as bevacizumab (AVASTIN™, Genentech): and (xi) pharmaceutically acceptable salts, acids or derivatives of any of the above.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In is another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN™ rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

A “dosing regimen” (or “therapeutic regimen”), as that term is used herein, is a set of unit closes (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, the term “immune checkpoint modulator” refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g. disinhibition). In some embodiments, an immune checkpoint modulator interferes with a signal for T cell anergy. In some embodiments, an immune checkpoint modulator reduces, removes, or prevents immune tolerance to one or more antigens.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The term “inflammation”, as used herein, refers to a local response to cellular injury that is characterize by capillary dilatation, leukocyte infiltration, redness, heat, and pain and that serves as a mechanism initiating the elimination of noxious agents and of damaged tissue. Inflammation is normally a self-limited process avoiding unnecessary organic damage. “Inflammatory disease” is a medical condition characterized by exaggerated or chronic inflammation. Autoimmune diseases are generally also inflammatory diseases. However, numerous inflammatory diseases are not consider autoimmune diseases. Examples of inflammatory diseases are Sepsis, Alzheimer, ankylosing spondylitis, arthritis, asthma, atherosclerosis, colitis, dermatitis, diverticulitis, fibromyalgia, hepatitis, irritable bowel syndrome, nephritis, Parkinson, Sclerosis multiple, bipolar disorder, autism, type-2 diabetes, osteoporosis and obesity.

As used herein, the term “kit” refers to any delivery system for delivering materials. Inclusive of the term “kits” are kits for both research and clinical applications. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides or liposomes. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

The term “modulator” is used to refer to an entity or agent whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

As used in this specification and the appended claims, the term “or” is generally employed in its sense including “and/or” unless the content clearly dictates otherwise.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

By “proliferative disease” or “cancer” as used herein is meant, a disease, condition, trait, genotype or phenotype characterized by unregulated cell growth or replication as is known in the art; including colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of its environment (e.g., treating the environment with an antibiotic effective against a bacterial bioform), alone or in combination with other therapies.

The term “sample” as used herein refers to a biological sample obtained for the purpose of evaluation in vitro. With regard to the methods disclosed herein, the sample or patient sample preferably may comprise any fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

The terms “subject”, “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates. Patients in need of therapy comprise those at risk of developing a certain condition, disease or disorder (e.g. due to genetic, environmental or physical attributes, such as for example, obesity). Patients in need of therapy also include those afflicted with a condition, disease or disorder. The diseases or disorders comprise, for example: autoimmune diseases, cancer, inflammatory diseases, neurological diseases or disorders, neuroinflammatory diseases or disorders, cardiovascular disease, obesity, diseases or disorders caused by infectious agents such as, for example, viruses, bacteria, fungi, prions, or parasites.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a. symptom of a disease (e.g., lessen the pain or discomfort), wherein such amelioration may or may not be directly affecting the disease (e.g., cause, transmission, expression, etc.).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press. 1986); B. Perbal, A Practical Guide To Molecular Cloning (1984); Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### BRIEF DESCRIPTION OF THE DRAWINGS

FIGS. 1A-1C are graphs demonstrating the kinetic profiles of mRNAs induced by incubation of human CD19⁺ B cells with IMT504. FIG. 1A: mRNAs induced at early times returning near to the basal level before the 22 h incubation time. FIG. 1B: mRNAs induced at early times and remaining at the maximal reached level at the 22 h incubation time. FIG. 1C: mRNAs whose level is increasing at the 22 h incubation time.

FIGS. 2A-2C are graphs demonstrating the kinetic profiles of interleukin secretion induced by human CD19⁺ B cells incubation with IMT504. FIG. 2A: IL10 secretion profile. FIG. 2B: IL8 secretion profile. FIG. 2C: IL35 secretion profile.

FIGS. 3A-3B: Cytometric analysis of CD19⁺B cells incubated for 36 h in the absence (control) or in the presence of IMT504. FIG. 3A: CD27 vs MUC1 markers, FIG. 3B: CD24 vs MUC1 markers, FIG. 3C: CD38 vs MUC1 markers and FIG. 3D: CD138 vs MUC1 markers. CD27 is a memory B cell marker, CD24 and CD38 are naïve B cell markers and CD138 is a plasma cell marker. MUC1 is a surface marker highly expressed upon CD19⁺B cell treatment with IMT504. The red circle indicate differences between control and IMT504 treated cells.

FIGS. 4A-4B are graphs showing the IMT504-treated lymphocyte B effects on the inflammatory response in the Corpus Callosum (CC) of CPZ-demyelinated rats. Quantification of Allograft Inflammatory Factor 1 (IBA1) (FIG. 4A) and the inflammatory marker CD68 (FIG. 4B) positive microglial cells by immunohistochemistry in the CC of CPZ and control animals 7 days after intravenous injection of 1×10⁵ SS-incubated or IMT504-incubated B lymphocytes. Results are expressed as positive cells by area.

FIGS. 5A-5B demonstrate the IMT504-treated lymphocyte B effects on mature oligodendrocytes in the Corpus Callosum (CC) of CPZ-demyelinated rats. FIG. 5A: Representative images of mature oligodendrocyte marker adenomatous polyposis coli (APC) and myelin-associated glycoprotein (MAG) immunohistochemistry in the CC of CPZ and control animals 7 days after intravenous injection of saline solution (SS), IMT504 (20 mg/kg body weight), 1×10⁵ SS-treated lymphocytes or 1×10⁵ IMT504-treated lymphocytes; cell nuclei visualized with Hoechst. Magnified images show MAG positive cells. FIG. 5B: Quantification of MAG positive cells. Results are expressed as positive cells by area.

### DETAILED DESCRIPTION

During a study on the effect of the single stranded, phosphorothioate, immunomodulatory oligonucleotide IMT504 (3) on the transcription profile of CD19⁺ B cells, it was found that 20 hours contact between the oligonucleotide and the cells, an abundant cell population with a remarkable transcriptome corresponding to a novel, powerful regulatory B cell was identified, termed herein “Breg-nov”.

### B Cells

B cells are lymphocytes that recognize antigens through a molecule called the B cell receptor (BCR). The BCR is a surface immunoglobulin (Ig) molecule that recognizes the antigen and is associated with two additional proteins, which transduce the signal. Upon encountering its antigen, a B cell begins a process of activation that leads to antibody secretion and memory formation regulated by interplay with antigen-activated T cells, dendritic cells (DCs), soluble factors, and in some cases follicular dendritic cells (FDCs). Both T and B lymphocytes can differentiate from naïve to memory cells, but only B cells have the capacity to fine-tune their antigen receptor structure to increase its specificity and affinity, giving rise to more effective antibodies. Beyond immunoglobulin secretion, B cells regulate the immune response by cytokine secretion and antigen presentation to T cells in the context of class II major histocompatibility complex (MHC) molecules. B cells have a positive role in priming adaptive CD4⁺ T cells, but not CD8⁺ T cells. The magnitude of CD4⁺ T-cell responses is reduced upon pathogen challenge in B-cell deficient or -depleted mice. B cells are also able to dampen T-cell driven immune responses, giving rise to the concept of regulatory B cells (Breg).

B cells produce cytokines in response to their environment. Several subsets of B cells have been reported to be able to suppress autoimmunity, including CD1d^{hi}CD5⁺ B cells and transitional B cells. All these B regulatory cells (Bregs) produce IL-10 to suppress immune responses. The function of Bregs depends on stimulation through BCR and CD40. In healthy persons. Bregs secrete IL-10 in response to CD40 engagement, whereas the equivalent population in patients with systemic lupus erythematosus (SLE) fail to do so. Bregs are also able to mediate immunosuppression through an IL-10-independent mechanism. Bregs secreting IL10 or transforming growth factor β (TGFβ) have been identified in other animal models of auto-immunity, cancer and infection, supporting the concept that these cells have an important role in maintaining peripheral tolerance. Recent studies have identified IL-35 as an additional effector molecule for Breg function. Some IL-35-producing Bregs express CD138 and Blimp-1. Thus, activated B cells and plasma cells play an important role in regulating immune responses. These Bregs not only harness autoimmunity but also restrain immune responses against microbial infection.

### Immune Cell Activating Compositions

IMT504 has demonstrated to be therapeutically effective when injected in mammals suffering a number of inflammatory and/or autoimmune disorders (3) and according to its properties, Breg-nov may be a key intermediary in the therapeutic effect of the IMT504 treatment. However, introduction of IMT504 in the mammal body may modify many other cells besides B cells and consequences of this are largely unknown. Regarding this, in toxicity preclinical studies, non-tolerable side effects were observed when injecting IMT504 in doses superior to 50 mg/Kg (4). Therefore, success in the treatment of at least some pathologies with IMT504 can be limited by safety reasons. An alternative to the IMT504 treatment by the parenteral route is the ex-vivo treatment of CD19⁺ B cells from a subject with IMT504 in order to generate a significant population of Breg-nov that may then be infused into the subject for therapeutic purposes. This procedure, has the advantage that IMT504 could be used at any concentration in order to optimize differentiation of CD19⁺ B cells to Breg-nov and then easily eliminated, for example by washing the cells, before cell reinfusion in the subject. Further advantage of the ex-vivo treatment of CD19⁺ B cells with IMT504 in order to obtain Breg-nov cells, is that these cells can be infused in a direct, short route to a given damaged organ (for example the coronary artery for the heart or the intrathecal route for the central nervous system), attaining an effective Breg-nov concentration directly into the damaged area. This will also minimize Breg-nov cells loss by mortality before reaching the damage organ/tissue.

An immunological disorder can be treated by contacting lymphocytes from the subject with the oligonucleotide of the invention (IMT504) “ex-vivo” and re-administering the activated cells to the subject. However, to be successful this treatment should have a sufficient number of cells with the appropriate phenotype are return to the patient. Thus, there remains a need for methods of producing highly active, phenotypically distinctive, Breg cells of an appropriated phenotype in adequate quantities as well as how using these cells in therapeutic procedures in order to obtain the best possible results.

In certain embodiments, a pharmaceutical composition comprises a therapeutically effective amount of a phosphorothioate oligonucleotide IMT504, having the sequence TCATCATTTTGTCATTTTGTCATT (SEQ ID NO: 1). In certain embodiments, a pharmaceutical composition comprises a therapeutically effective amount of a phosphorothioate oligonucleotide having at least a 50% sequence identity to SEQ ID NO: 1. In certain embodiments, a pharmaceutical composition comprises a therapeutically effective amount of a phosphorothioate oligonucleotide has at least a: 60%, 70%, 75%, 80%, 85%, 90%, 91%, 92%, 93%, 94%, 95%, 96%, 97%, 98%, 99%, or at least a 99.9% sequence identity to SEQ ID NO: 1. The term “percent sequence identity” refers to the degree of identity between any given query sequence and a subject sequence.

In certain embodiments, a pharmaceutical composition comprises a therapeutically effective amount of a phosphorothioate oligonucleotide IMT504, having the sequence TCATCATTTTGTCATTTTGTCATT (SEQ ID NO: 1) and a second agent. The second agent can be, for example, a chemotherapeutic agent, a cytokine, a chemokine, an anti-inflammatory agent, non-steroidal anti-inflammatory drugs with analgesic, antipyretic and anti-inflammatory effects, an immune modulator, an immunotherapeutic, growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

Modified or Mutated Nucleic Acid Sequences: In certain embodiments, the nucleic acid sequence of SEQ ID NO: 1 may be modified or derived from a native nucleic acid sequence, for example, by introduction of mutations, deletions, substitutions, modification of nucleobases, backbones and the like. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH,—N(CH₃)—O—CH₂ [known as a methylene(methylimino) or MMI backbone], CH₂—O—N(CH₃)—CH₂, CH₂—N(CH₃)—N (CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequence of SEQ ID NO: 1 may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil. 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

### Methods and Uses Thereof

The invention provides a method for preparing B-cells that produce: Neudecin, Pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 and Prostaglandin E2 synthase (here in named Breg-nov). This method comprises contacting one or more CD19⁺ B cells, ex-vivo, with the phosphorothioate oligonucleotide IMT504, having the sequence TCATCATTTTGTCATTTTGTCATT (SEQ ID NO: 1), for at least 20 or more hours under conditions to provide one or more Breg-nov cells. In certain embodiments, a phosphorothioate oligonucleotide has at least a 50% sequence identity to SEQ ID NO: 1. In certain embodiments, the phosphorothioate oligonucleotide has at least a: 60%, 70%, 75%, 80%, 85%, 90%, 91%, 92%, 93%, 94%, 95%, 96%, 97%, 98%, 99%, or at least a 99.9% sequence identity to SEQ ID NO: 1. The term “percent sequence identity” refers to the degree of identity between any given query sequence and a subject sequence.

In addition, the invention provides a method of suppressing-autoimmunity or suppressing acute or chronic inflammation or repairing a damaged organ or tissue, or cancer in a mammal by reinfusion of Breg-nov cells prepared in vitro from B19⁺ B cells extracted from the mammal according to the above mentioned inventive method. In certain embodiments, extracellular vesicles or IL-35 are obtained from the Breg-nov culture supernatant and used for therapeutic purposes.

Further, in certain embodiments, a method of modulating an immune response comprises contacting one or more immune cells with the phosphorothioate oligonucleotide SEQ ID NO: 1 (IMT504), variants, derivatives or fragments thereof. In certain embodiments, the phosphorothioate oligonucleotide has at least a 50% sequence identity to SEQ ID NO: 1. In certain embodiments, the phosphorothioate oligonucleotide has at least a: 60%, 70%, 75%, 80%, 85%, 90%, 91%, 92%, 93%, 94%, 95%, 96%, 97%, 98%, 99%, or at least a 99.9% sequence identity to SEQ ID NO: 1.

In certain embodiments, a method of regulating an immune response, comprises contacting one or more cells, with a phosphorothioate oligonucleotide having at least a 50% sequence identity to SEQ ID NO: 1. In certain embodiments, the phosphorothioate oligonucleotide has a sequence of at least 70% to SEQ ID NO: 1. In certain embodiments, the one or more cells comprise immune cells. In certain embodiments, the immune cells comprise: B cells, T cells, antigen presenting cells, chimeric antigen receptor-T cells (CAR-T) or combinations thereof.

In certain embodiments, the cells are autologous cells, comprising: autologous, allogeneic, haplotype matched, haplotype mismatched, haplo-identical, xenogeneic, cell lines or combinations thereof.

In certain embodiments, the cells are stem cells.

In another preferred embodiment, a method of treating cancer comprises obtaining immune cells; culturing and contacting the immune cells with a composition comprising a phosphorothioate oligonucleotide having at least a 50% sequence identity to SEQ ID NO: 1 and/or a tumor antigen, and administering the immune cells to the subject, thereby treating cancer.

In certain embodiments, the method further comprises administering one or more chemotherapeutic agents and/or a phosphorothioate oligonucleotide having at least a 50% sequence identity to SEQ ID NO: 1. In certain embodiments, the method comprises administering to the subject one or more chemotherapeutic agents and/or radiotherapy and/or surgery.

In certain embodiments, a composition comprises a therapeutically effective amount of a phosphorothioate oligonucleotide having at least a 50% sequence identity to SEQ ID NO: 1 (IMT504), one or more anti-inflammatory agents, other therapeutics, immunosuppressive agents, chemotherapeutic agents or combinations thereof.

In adult mammals, B-lymphocytes develop in the bone marrow from hematopoietic precursor cells up to immature B cells that leave the bone marrow travelling to the spleen, were they differentiate into naïve, follicular and marginal zone B cells. Follicular B cells are activated by antigen binding differentiating, in the germinal centers, into memory B cells or antibody secreting plasma cells. In the human circulating blood, approximately two-thirds of the B cells are naïve B-lymphocytes and one-third memory B cells. In addition to their well-known role in humoral immunity, B lymphocytes contribute directly to cellular immunity via at least three mechanisms: I—Serving as antigen-presenting cells (APCs) that enhance T lymphocyte-mediated immunity; II—Functioning as cellular effectors that produce inflammatory cytokines; and III—Differentiating into regulatory B cells (Breg) characterized by anti-inflammatory modulation of the immune response (5). Breg, could downregulate excessive immune and inflammatory responses through inhibitory cytokines, such as is interleukin 10 (IL-10), interleukin 35 (IL-35) and transforming growth factor beta (TGF-β). Accordingly, they could be useful for cell therapy procedures and successful proof of concept studies in animal models of disease have been reported (1,6). However, Bregs are a small subset of B cells; therefore, its collection for use in cell therapy medical procedures is extremely difficult. Thus, development of efficient methods to obtain large amounts of Bregs in vitro is very important.

Breg cells can be divided into several functionally distinct subsets that are capable of inhibiting inflammatory responses and inducing immune tolerance (1). Thus, in the context of the invention, a “regulatory B-cell” is a B-cell that produces: Neudecin (NENF), Granulin (GRN), Epidermal growth factor (EGF), Wnt family member 8B (Wnt8B), Secreted frizzled related protein (SFRP5), Epstein-Barr virus induced 3 (EBI3), Apurinic/Apyrimidinic endonuclease 1 (APEX1, Phospholipid transfer protein (PLTP), Mucin 1 (MUC1) and Prostaglandin E2 synthase (PTGES2). In one embodiment, the inventive method comprises contacting one or more CD19⁺ B cells, ex-vivo, with the phosphorothioate oligonucleotide IMT504, having the sequence TCATCATTTTGTCATTTTGTCATT (SEQ ID NO: 1), for at least 20 hours under conditions to provide one or more B cells that produce: Neudecin, Pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 and Prostaglandin E2 synthase. These B reg cells were named Breg-nov cells. In order to understand the general properties of Breg-nov cells, a brief description of relevant proteins produced by these cells are now described:

Neudesin (NENF gene product) is a secreted protein widely expressed in various tissues including brain, adipose tissue, heart, lung, and kidney at postnatal stages. The expression profile and activity of Neudesin indicate that it plays unique roles in neural cell proliferation and neuronal differentiation (7). An extensive behavioral characterization of Neudesin KO mice revealed anxiety-like behavior and a role of neudesin in maintaining the hippocampal anxiety circuitry was propose (8).

Pro-Granulin (GRN gene product) is a secreted protein widely express in epithelia, bone marrow, immune cells, solid organs and the nervous system (9). Pro-Granulin binds receptors for tumor necrosis factor-α (TNF-α); and inhibits downstream TNF-α signal transduction. The anti-inflammatory effects of ProGranulin are evident in grn−/− knockout mice which mount highly exaggerated inflammatory responses (10). A large number of studies have confirm the protective role of Pro-Granulin in inflammatory disorders (11,12,13).

Galectin 3 (LGALS3 gene product) is a secreted protein presenting multifaceted actions in the innate immune responses against pathogens (14). Lately, numerous studies have demonstrated important effects of Galectin 3 on survival, migration, and immunomodulatory actions of Mesenchymal Stem Cells (MSCs) and Hepatic Progenitor Cells (HPCs) (15,16,17).

Epidermal growth factor (EGF gene product) is a secreted growth factor that plays an important role in proliferation, differentiation and migration of a variety of cell types (18). Over the last decade, epidermal growth factor has emerged as a powerful regulator of stem cells in different tissues, such as neural stem/progenitor cells (19), neural crest stem cells (20), cardiac stem cells (21), bone marrow stromal cells (MSCs) (22), gut stem cells (23) and keratinocyte stem cells (24).

Wnt family member 8B (Wnt8B gene product) is a secreted protein that signal through the canonical Wnt signaling pathway. This pathway, have been identify and link to signaling regulation, stem cell functions, and adult tissue homeostasis (25). The Wnt signaling cascade has been identify as a regulator of self-renewal and proliferation among a variety of stem and progenitor cell populations including neural stem/progenitor cells, epithelial stem cells and bone marrow MSCs (26,27,28,29).

Secreted Frizzled Related Protein 5 (SFRP5 gene product) is a secreted protein that has anti-inflammatory effects by suppression of the non-canonical, pro-inflammatory Wnt5a/JNK signaling pathway (30,31,32).

Epstein-Barr Virus Induced 3 (EBI3 gene product) is a protein that serves as subunit of two immunosuppressant/anti-inflammatory cytokines: IL27 and IL35 (33). Secretion of IL35 by some B regulatory cells in mice has recently being reported (34,35). Several reports has shown that IL35 mediates protection in experimental immune disorders (36,37,38,39).

Apurinic/Apyrimidinic Endodeoxyribonuclease 1 (APEX1 gene product) is a protein with a central role in the cellular response to oxidative stress and also, as a secreted form, inhibit pro-inflammatory signaling of TNFα via disruption of the TNFR1 receptor (40,41,42).

Phospholipid Transfer Protein (PLTP gene product) is a secreted plasma protein that facilitates bacterial LPS clearance suppressing NFκB activation induced by this LPS endotoxin (43,44).

Mucin 1 (MUC 1 gene product) is a cell-surface associated protein that specifically inhibits activation of the NLRP3 inflammasome, limiting inflammation by bacteria (45,46).

Integrin Subunit Alpha 2 (CD49B) (ITGA2 gene product) is a cell surface protein that in combination with another cell surface protein called Lymphocyte Activating 3, identify a very potent population of T regulatory cells (Tr1s) that Suppress NLRP3 Inflammasome Activation (47,48).

Prostaglandin E Synthase 2 (PTGES2 gene product) is an enzyme that catalyzes the synthesis of prostaglandin E2 (PGE2), a secret lipid that promotes differentiation of macrophages and monocytoid dendritic cells to an anti-inflammatory phenotype (3).

On the other hand, Breg-nov cells produces a number of anti-oxidative stress proteins (Table 6) that confers to the Breg-nov cells a phenotype of resistance to harsh conditions typical of inflamed tissues were these cells should mainly locate for action (49). On the other hand, Breg-nov cells produce a number of mitochondrial proteins (Table 7) and extracellular vesicles associated proteins. It is well known that cell components can be transferred between cells by means of externalized vesicles, in order to help recovering of damaged organs/tissues (46,47,48,49).

Collectively, late induced proteins induced by incubation of B cells with IMT504, indicate that the generated Breg-nov are strongly immunosuppressive, anti-inflammatory and pro-organ/tissue reparatory cells.

The immunomodulatory IMT504 agent used in the inventive method in order to induce CD19 B cells to differentiate into Breg-nov cells is a phosphorothioate oligonucleotide, 24 nucleotides long and with the following sequence: TCATCATTTTGTCATTTTGTCATT (SEQ ID NO: 1). When injected in animals, IMT504 induces a significant expansion of MSCs in the blood. In addition, IMT504 injection resulted in a marked improvement in animal models of neuropathic pain, osteoporosis, diabetes and sepsis (3). These facts led to the hypothesis that IMT504 may act through the well know anti-inflammatory and tissue reparatory action of MSCs (3). It was surprisingly found that incubation of CD19 B cells, in vitro, with IMT504 results after 20 hours or more in the development of an abundant population of B cells that, according their capacity to secret proteins with anti-inflammatory and/or tissue reparatory activity, can be classify as regulatory B cells. The phenotype of these cells, named by us Breg-nov, can explain many if not all the IMT504 anti-inflammatory and tissue reparatory activities of IMT504. On the other hand, some of the proteins secreted by Breg-nov are able to stimulate proliferation and/or differentiation of MSCs (e.g. Galectin 3, Epidermal Growth Factor and Wnt family member 8B). Therefore, Therapeutic activity of IMT504 can now be better explained by a primary action on circulating CD19⁺B cells followed of Breg-nov generation, and followed of secondary connections with other cells (e.g. MSCs, macrophages and monocytoid dendritic cells) that acquire anti-inflammatory and/or tissue repair phenotypes through the action of Breg-nov secreted and/or cell surface and/or enzymatic protein products.

Upon introduction of IMT504 in a mammal body, it may modify many other cells besides B cells and consequences of this are largely unknown. Regarding to this, in toxicity preclinical studies, non-tolerable side effects were observe injecting IMT504 in doses superior to 50 mg/Kg (4). Therefore, success in the treatment of at least some pathologies with IMT504 are limit, because of safety reasons, to the use of doses lower to 50 mg/Kg. An alternative to the IMT504 treatment by the parenteral route is the ex-vivo treatment of CD19⁺B cells, from a subject, with IMT504 in order to generate a significant population of Breg-nov that could then be reinfuse into the subject for therapeutic purposes. This procedure, have the advantage that IMT504 could be use at any concentration in order to optimize differentiation of CD19⁺B cells to Breg-nov and then easily eliminated, for example by washing the cells, before cell reinfusion in the subject. Many other procedures to separate cells from small molecules, like IMT504, are well known in the art and can be used, instead of washing, in the inventive method, providing that they do not damage the cells. Another advantage of the use of the Breg-nov cells in a therapeutic procedure, is that these cells can be infused directly into a short route to a given damaged organ (e.g. the coronary artery for the hart or the intrathecal route to reach the central nervous system) in order to seed an effective Breg-nov concentration directly into the damaged area. This will also avoid cell loss by mortality before reaching the target. Injection of IMT504 in this same way may not be as effective because rapid distribution in the body own to its high solubility and/or absence of abundant B bell target cells in the damaged area. Another, possible advantage of the Breg-nov infusion treatment vs IMT504 injection is avoiding possible allergies to the phosphorothioate oligonucleotides.

The inventive method to obtain Breg-nov involves contact of one or more B-cells, preferably CD19⁺ B cells, with an appropriate amount of IMT504 “ex vivo”. “Ex vivo” refers to methods conducted with cells, tissues or organs outside an organism minimizing alterations of the natural conditions present inside the organism.

Suitable methods for purification, culture and characterization of B-cells are well known in the art (see for example: protocol-online.org/prot/CellBiology/CellCulture/index.html).

The one or more CD19⁺B cells preferably are obtained from a mammal, more preferably a mouse, a rat and most preferably a human. The one or more CD19⁺B-cells preferably are CD19⁺ B primary cells. The term “primary cell” refers to a cell that is isolated directly from living tissue. In the context of the invention, primary B cells can be isolated from peripheral blood of a patient suffering from, for example, cancer, an autoimmune and/or inflammatory disease.

The inventive method of suppressing-autoimmunity or suppressing acute or chronic inflammation or repairing a damaged organ or tissue in a mammal using Breg-nov cells, comprises administering to a mammal Breg-nov that produce, Neudecin, Pro-Granulin, Galectin 3, Epidermal Growth Factor, Wnt Family Member 8B, Secreted Frizzled Related Protein, Epstein-Barr virus Induced 3, Apurinic/Apyrimidinic Endonuclease 1, Phospholipid Transfer Protein, Mucin 1, Integrin Subunit Alpha 2 and Prostaglandin E2 synthase, whereby autoimmunity or acute or chronic inflammation or organ/tissue damage is suppressed in the mammal including humans.

During trauma, cancer, autoimmune disease and/or inflammatory disease, tissues and organs result damaged. Even after resolution of inflammation accumulated, damage and fibrosis may severally limit organ or tissue functionality. On the other hand, wounds or bums may be life threatening. In all this cases rapid tissue damage resolution, preserving functionality is highly desirable.

Thus, in one embodiment, the inventive method is used to treat an autoimmune disease, an inflammatory disease, or tissue damage in a mammal including a person. As used herein, the term “treatment,” refers to a procedure to obtain a desired pharmacologic effect. Preferably, the effect is therapeutic, that is, the effect partially or completely cures a disease.

Exemplary autoimmune diseases which may be treated by the present method include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease and anti-helper T lymphocyte autoimmunity.

Examples of autoimmune rheumatoid diseases include, but are not limited to rheumatoid arthritis and ankylosing spondylitis.

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Examples of autoimmune gastrointestinal diseases include, but are not limited to, chronic inflammatory intestinal diseases, celiac disease, colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis, primary biliary cirrhosis and autoimmune hepatitis.

Examples of autoimmune neurological diseases include, but are not limited to, multiple sclerosis, Alzheimer's disease, myasthenia gravis, neuropathies, motor neuropathies, Guillain-Barre syndrome and autoimmune neuropathies, myasthenia, Lambert-Eaton myasthenic syndrome, paraneoplastic neurological diseases, cerebellar atrophy, paraneoplastic cerebellar atrophy and stiff-man syndrome, non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies, dysimmune neuropathies, acquired neuromyotonia, arthrogryposis multiplex congenita, neuritis, optic neuritis and neurodegenerative diseases.

Examples of autoimmune muscular diseases include, but are not limited to, myositis, autoimmune myositis and primary Sjogren's syndrome and smooth muscle autoimmune disease.

Examples of autoimmune nephric diseases include, but are not limited to, nephritis and autoimmune interstitial nephritis.

Examples of autoimmune diseases related to reproduction include, but are not limited to, repeated fetal loss.

Examples of autoimmune connective tissue diseases include, but are not limited to, ear diseases, autoimmune ear diseases and autoimmune diseases of the inner ear.

Examples of autoimmune systemic diseases include, but are not limited to, systemic lupus erythematosus and systemic sclerosis.

In certain embodiments, the compositions are administered to patients to prevent or treat an acute inflammatory disease, a chronic inflammatory disease, a neurodegenerative disease, a malignant tumor, or a benign tumor.

In certain embodiments of the present invention, the inflammatory disease comprises: psoriasis, rheumatoid arthritis (RA), Morbus Bechterew, multiple sclerosis (MS), systemic lupus erythematosus (SLE), Behcet's disease, uveitis, Sjogren syndrome, an inflammatory bowel disease (IBD), asthma, chronic obstructive pulmonary disease (COPD), neuropathic pain, atopic dermatitis, or allergy.

Exemplary inflammatory diseases which may be treated by the present method include, but are not limited to, chronic inflammatory diseases and acute inflammatory diseases.

Inflammatory diseases associated with hypersensitivity: Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, includes, for example, asthma.

Type II hypersensitivity include, but are not limited to, rheumatoid diseases, rheumatoid autoimmune diseases, rheumatoid arthritis (Krenn V. et al., Histol Histopathol 2000 Jul. 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus, 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome; anti-factor VIII autoimmune disease (Lacroix-Desmazes S. et al., Semin Thromb Hemost, 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis; antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (492); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin. Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, T_{H}1 lymphocyte mediated hypersensitivity and T_{H}2 lymphocyte mediated hypersensitivity.

In another embodiment, the inventive method is used as a prophylactic procedure to prevent an undesirable event, i.e., rejection of an organ, tissue or cells transplant. Regarding to this, the inventive method comprises administering a “prophylactically effective amount” of Breg-nov to a person that has received a transplant in order to avoid rejection. “Prophylactically effective amount” refers to an amount effective, at dosages and for periods of time necessary, to attain a desired prophylactic result (e.g., prevention of graft vs. host disease).

In certain embodiments, a method of treating a subject comprising administering the phosphorothioate oligonucleotide in combination with one or more therapeutic agents.

As used herein, the term “combination” embraces groups of compounds or non-drug therapies useful as part of a combination therapy. Such combination treatment is achieved by way of the simultaneous, sequential, or separate dosing of the individual components of the treatment. In certain examples, a composition of the invention is conjointly administered with one or more ant-inflammatory agents, chemotherapeutics, other therapeutics or combinations thereof.

In certain embodiments, a composition of the invention is administered in combination with a non-steroidal anti-inflammatory. Suitable non-steroidal anti-inflammatory compounds include, but are not limited to, piroxicam, diclofenac, etodolac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib.

According to the invention, Breg-nov cells can be administered to a mammal, including a person, using standard cell transfer techniques. Examples of these techniques include autologous cell transplant, allogeneic cell transplant, and hematopoietic stem cell transplant. In a preferred embodiment, a composition comprising Breg-nov cells is transplant to a mammal via adoptive transfer methods (50). The Breg-nov cells can be administer to a human or other mammal in a suitable amount in order to achieve a desirable therapeutic effect. A usual dose of cells administered to a person or other mammal can be in the range of one million to 100 million cells. Though, amounts below or above this range are within the scope of the invention. For example, the dose of Breg-nov cells can be of about 1 million to about 50 million cells (e.g. 25 million cells), preferably of about 10 million to about 100 million cells (e.g. 70 million cells). The Breg-nov cells dose can be administered once or can be administered daily for a number of consecutive days (e.g. for 5 consecutive days) or can be administered one every other day (e.g. 5 doses administered one every other day) or using any combination of number of doses and periods of time as necessary to reach a desirable therapeutic effect.

The inventive method can be perform alone or in combination with other standard therapies. For example, infusion of the Breg-nov cells can be used in combination with immunosuppressive, anti-inflammatory or tissue repair agents for the treatment or prevention of a disease disclosed herein. Furthermore, Breg-nov can be associated with devises aimed to repair injured organs and tissues including bones (e.g. patches for treatment of bums, patches for treatment of ulcers or dental implants).

Anti-inflammatory and tissue repair cells like Treg, MSC or regulatory monocytoid dendritic cells release to the milieu vesicles that can perform many of the biological tasks attributed to the mother cell (51). In fact, several of these cell tasks are dependent of the release of vesicles (the smaller named exosomes) which facilitates cell to cell communication by transference of proteins or even complex cellular structures like mitochondria and/or proteasomes (52). Therapeutic capability of these cell derived vesicles, is currently an intensive field of investigation (53, 54). Example 2, illustrates that differentiation of CD19⁺B cells to Breg-nov is accompanied of strong transcription activation of genes codifying for vesicular associated proteins (Table 7) and for mitochondrial proteins (Table 6). Breg-nov derived vesicles released to the cell milieu can be recovered from it by means of methods, well-known in the art (55).

Another important product of the Breg-nov is IL35 (Example 3) which also possesses important therapeutic activities (56). IL-35 can be recovered from biologic fluids by well-known in the art protein purification procedures (57).Thus, Breg-nov extracellular vesicles or IL35 can be obtained using the method of this invention by addition of a few well known in the art recovering and purification steps, and these derived combined methods for producing them are also in the scope of this invention.

### Recombinant Constructs and Delivery Vehicles

Recombinant constructs are also provided herein and can be used to transform cells in order to express the isolated nucleic acid sequences embodied herein. A recombinant nucleic acid construct comprises promoter operably linked to a regulatory region suitable for expressing SEQ ID NO: 1 or variants thereof.

It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fitly nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques. 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The isolated nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLG (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising SEQ ID NO: 1 or variants thereof as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to therm-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more vectors encoding the isolated nucleic acids embodied herein. In some embodiments, the methods of the invention can be applied ex vivo. That is, a subject's cells can be removed from the body and treated with the compositions in culture to excise, and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

### Combination Therapy

Compositions of the invention may be combined in a pharmaceutical combination formulation, or dosing regimen as combination therapy, with a second compound, for example, chemotherapeutic agents, agents used in the treatment of autoimmune diseases, etc. The second compound of the pharmaceutical combination formulation or dosing regimen preferably has complementary activities to the compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak, or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered with a vaccine (e.g., anti-cancer vaccine) therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one that inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine;. Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinflunine; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

In certain embodiments, the composition comprises SEQ ID NO: 1, or B cells which have been cultured ex vivo and one or more second, third, fourth, fifth etc., agents

With respect to treatment of autoimmune disease, excessive and prolonged activation of immune cells, such as T and B lymphocytes, and overexpression of the master pro-inflammatory cytokine tumor necrosis factor alpha (TNF), together with other mediators such as interlukin-6 (IL-6), interlukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying antirheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases. Other more powerful immunosuppressant drugs, such as methotrexate, cyclophosphamide, and azathioprine can also be used in combination therapies.

According to the methods of the invention, the agents of the invention may be administered prior to, concurrent with, or following the other therapeutic compounds or therapies. The administration schedule may involve administering the different agents in an alternating fashion. In other embodiments, the agent may be delivered before and during, or during and after, or before and after treatment with other therapies. In some cases, the agent is administered more than 24 hours before the administration of the second agent treatment. In other embodiments, more than one anti-proliferative therapy or an autoimmune therapy may be administered to a subject. For example, the subject may receive the agents of the invention, in combination with both surgery and at least one other anti-proliferative compound. Alternatively, the agent may be administered in combination with more than one anti-cancer drug.

### Pharmaceutical Compositions

In certain embodiments, the present invention provides for a pharmaceutical composition comprising a SEQ ID NO: 1 as identified herein. The composition can be suitably formulated and introduced into a subject or the environment of a cell (e.g., immune cell, lymphs, a neoplasia, a cancer cell or a tumor) by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Compositions of the present invention are administered to subjects in a variety of routes including but not limited to: oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration. The composition may be administered directly into the cancerous tumor, or in some embodiments can be administered to the immune cell.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

As defined herein, a therapeutically effective amount of SEQ ID NO: 1 composition of the invention targeting a disease or disorder (i.e., an effective dosage) depends on the target disease or disorder selected. For instance, single dose amounts of a composition of the invention targeting a disease or disorder in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered.

A therapeutically effective amount of the compound of the present invention can be determined by methods known in the art. The therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for example, 50-500 mg/day, 50-300 mg/day and 100-200 mg/day.

Administration may be a single dose, multiple doses spaced at intervals to allow for an immunogenic response to occur, once a day, twice a day, or more often, and may be decreased during a maintenance phase of a disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions embodied herein, can include a single treatment or, optionally, can include a series of treatments.

The compositions of the invention could also be formulated as nanoparticle formulations. The compounds of the invention can be administered for immediate-release, delayed-release, modified-release, sustained-release, pulsed-release and/or controlled-release applications. The pharmaceutical compositions of the invention may contain from 0.01 to 99% weight-per volume of the active material. For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the harrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including to liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in a method of the invention, the therapeutically effective dose can be estimated initially from cell culture assays. A dose may be formulated in animal models to achieve a circulating plasma concentration range that includes the IC₅0 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an agent (i.e., an effective dosage) depends on the agent selected. For instance, single dose amounts of an agent in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered.

A therapeutically effective amount of the compound of the present invention can be determined by methods known in the art. In addition to depending on the agent and selected/pharmaceutical formulation used, the therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, optionally, can include a series of treatments.

It can be appreciated that the method of introducing an agent into the environment of a cell will depend on the type of cell and the makeup of its environment. Suitable amounts of an agent must be introduced and these amounts can be empirically determined using standard methods. Exemplary effective concentrations of an individual agent in the environment of a cell can be 500 millimolar or less, 50 millimolar or less, 10 millimolar or less, 1 millimolar or less, 500 nanomolar or less, 50 nanomolar or less, 10 nanomolar or less, or even compositions in which concentrations of 1 nanomolar or less can be used.

The pharmaceutical compositions can be included in a kit, container, pack, or dispenser together with instructions for administration.

The following examples further illustrate the invention.

### EXAMPLES

### Example 1

### Materials and Methods

a) Microarray Studies

B Lymphocytes purification: Blood samples from healthy donors were obtained from the hematological division of the Hospital Aleman, (Buenos Aires, Argentina). Heparin was used as anticoagulant. Periphery Blood Mononuclear Cells (PBMC) were isolated by using the Ficoll-Hypaque (GE-Healthcare, BioScience, AB, Uppsala, Sweden) density gradient centrifugation technique. Briefly, blood samples ½ dilute in RPMI 1640 medium (Gibco, Thermo Fisher Scientific Inc. Waltham, Mass., USA) supplemented with 2.0 mM L-glutamine, 50.0 μg/ml gentamicin and 25 mM HEPES, were centrifuged at 1000×g for 40 min at 20° C. Pelleted PBMC were washed and suspended in medium supplemented with 10% FCS (Invitrogen, Thermo Fisher Scientific Inc. Waltham, Mass., USA).

CD19 B lymphocytes were purified from PBMC using CD19 Microbeads (MACS Order No 130-050-301, Miltenyi Biotec, Germany). Cell purity was >96% according to flow cytometric assays.

Cell Treatments and Cultures: Purified CD19 B cells cultures were carried out in 48 well plates, containing 0.5 ml of a cell suspension containing 2×10⁶ cells/ml. Cell were stimulated by incubation with IMT504 at a final concentration of 1.5 μg/ml. Control cells were incubated without IMT504. Samples for analysis were collect at 2, 4 and 22 hours.

RNA Extraction: Total RNA was extracted from five independent CD19 B cells cultures, using the RNeasy Mini Kit (QIAGEN Inc, Germantown, Md., USA). Total extracted RNA was pool and 1 μg of this pool use in the following reactions.

Microarrays and Reactions: CodeLink Uniset Human 20K I Bioarrays (Amersham Biosciences Limited, Buckinghamshire, UK) were used, following the instructions and recommendation described in the CodeLink Gene Expression System Manuals. Briefly, the assessment of concentration and quality of the total RNA sample were performed by spectrophotometry and electrophoresis in 1.2% agarose gels. Synthesis of first-strand cDNA (2 hours at 42° C.) was carried out using 1 μg of total RNA from CD19 B cells and a T7 oligo(dT) primer. Known quantities of particular bacterial mRNAs were included in the reactions as controls to estimate the mRNAs mass. Synthesis of the second-strand cDNA (2 hours at 16° C.) was perform using the total first-strand cDNA product. Purification of double-stranded cDNA was perform using the QIAQUICK™ PCR Purification Kit (QIAGEN Inc, Germantown, Md., USA).

Synthesis of cRNA (37° C. for 14 hours) was performed by IVT (In vitro Transcription) including biotinylated UTP (10 mM biotin-11-UTP, PerkinElmer. Waltham, Mass., USA) as one of the four ribonucleotides, and the T7 polymerase. The recovery of biotin-labelled cRNAs was carry out using the RNeasy Mini Kit (QIAGEN Inc, Germantown, Md., USA).

10 μg of cRNA were fragmented (94° C. for 20 minutes) and denatured before the hybridization reaction. Hybridization was performed placing the slides on a shaker plate and rotating at 300 rpm. Incubation was for 18 hr at 37° C. Several post-hybridization washes were carried out and for detection; incubation with Cy5-Streptavidin was used. After several washes including one with 0.1×SSC/0.05% TWEEN™ for 30 seconds, the slides were dried by centrifugation and scanned with the arrayWoRx™ “e” microarray scanner (APPLIED PRECISION, INC, Washington, USA).

Data and Statistical Analysis: Data from arrays were obtain using The CodeLink Expresión Análisis v4.1 software (Copyright Amersham Biosciences 2004) and the spot quality was evaluated with the same computer program. Only spots tagged as “Good” were included in this analysis.

Behavior of specific genes was studied using the Significance Analysis of Microarrays (SAM) software (Trustees of Leland Stanford Junior University, All Rights Reserved). The identification of significant modified genes was based on the concept of the false discovery rate (FDR) (1; 2) and the q value (3). A FDR of 10% was use in this analysis. Furthermore, gene induction induce by the IMT504 treatment was consider significant if the increment (or decrement) in the mRNA level, for a given gene, was superior to three fold.

b) Cytological Assays

B Lymphocytes purification: For cytological and cytokine secretion assays B cells were purified from human PBMCs using the B “CELL ISOLATION KIT HUMAN II MACS (130-091-151 Miltenyi Biotec, Bergish Gladbach, Germany)”. Cell purity was 98% according to flow cytometric assays.

Flow cytometry: For flow cytometry analysis, 1×10⁶ B cells were cultured in 96-well round bottom plates (100 μl cell suspension per well) at 37° C. in a 5% CO₂ humidified atmosphere for elected times as stated. Cell stimulation was achieved by addition of 6 μg/ml IMT 504. Control cells were incubated in the absence of IMT504.

After incubation, cells were harvested and stained, as recommended, with anti-CD24 PE-Vio 615conjugated (130-112-664; clone REA832; recombinant human IgG1; Milteny Biotec), anti-CD38 PE Vio770 conjugated (130-108-838; clone REA572; recombinant human IgG1; Milteny Biotec), anti-CD227 (Muc-1) APC conjugated (130-106-784 clone REA448, recombinant human IgG1; Milteny Biotec) in order to identify cell populations. All antibodies were incubated in the dark, at 4° C. for 10 min. After this, cells were washed with RPMI 1640 medium.

The samples were fixed in formaldehyde 1% at 4° C. for 15 min and washed with PBS. At least 5×10⁵ events were acquired using a FACSAria II cytometer (Becton Dickinson Immunocytometry Systems, San Jose, Calif., USA). Cell debris and dead cells were excluded from the analysis according to the scatter signal analysis. Viability was more than 85% after 72 h incubation as tested by supra-vital staining. Data were analyzed using the computer program FlowJo 7.6.

c) Cytokine Secretion Assays

IL-10 assay: B cell (1×10⁶ cells/well) were cultured as described above. After incubation, supernatants were collected, and IL-10 levels measured by ELISA (Human IL-10 ELISA Set/RUO; 555157 BD Biosciences, Brand OptEIA™).

Briefly, 96-well microliter plates (359454 NUNC) were coated with capture antibody anti-IL-10 over night at 4° C. After this, cell supernatant was aspirated and cells washed 3 times with wash buffer. After this, wells were incubated with RPMI 1640 medium supplemented with 10% (v/v) heat-inactivated FCS at room temperature for 1 h. IL10 standard dilutions and culture samples were then incubated in the coated wells for 2 hours at room temperature. Microliter plates were washed and IL-10 detected calorimetrically using a Working Detector (Detection Antibody+SAv-HRP reagent) for 1 hour at room temperature. Finally, substrate solution was added to each well and microplates were incubated during 55 min at room temperature in the dark. The reaction was stopped by the aggregate of stop solution and microplates read at 450 nm within the next 30 min with λ correction at 570 nm. Detection limit of these assays was 31.25 pg/ml. All assays were performed in duplicates.

IL-35 assay: iL-35 levels was measured by a human interleukin 35 (IL-35) ELISA Kit (EKC40941 Biomatik). Briefly, the standard dilutions and culture samples were incubated in assay plates 2 h at 37° C. After this, the plates were washed, anti-CD35 Biotin-antibody added and incubation proceeded for 1 h at 37° C. Plates were washed 5 times and incubated with Avidin-Horseradish Peroxidase solution for 1 h at 37° C. Finally, a substrate (TMB) solution was added to each well and the plates incubated during 15 m at 37° in dark. The reaction was stopped by the aggregate the stop solution and the plates were read at 450 nm within the next 5 min with correction 570 nm. The detection limit of these assays was 62.25 pg/ml.

### Example 2

### Transcriptome Analysis of CD19⁺ B Cells Incubated with IMT504

In vitro incubation of purified human CD19⁺ B cells with IMT504, analyzed at different times (2, 4 and 22 h) results in the induction of mRNAs corresponding to several genes (Table 1). All gene information can be found at the GeneCard database (genecards.org).

TABLE 1
Induce genes in CD19⁺ B cells incubated with IMT504

| | | Gene Expression | | |
|---|---|---|---|---|
| Gene | | (Fold Change) | | |
| Identifier | Description | 2 Hs | 4 Hs | 22 Hs |
| ACADVL | acyl-Coenzyme A | 0.69 | 1.42 | 4.59 |
| | dehydrogenase, very long | | | |
| | chain (ACADVL) | | | |
| ACO1 | aconitase 1, soluble (ACO1) | 0.75 | 1.19 | 3.02 |
| ACY1 | aminoacylase 1 (ACY1) | 1.03 | 1.49 | 5.13 |
| ACYP2 | acylphosphatase 2, muscle type | 1.50 | 1.72 | 3.16 |
| | (ACYP2) | | | |
| ADA | adenosine deaminase (ADA) | 0.70 | 2.25 | 7.14 |
| ADSL | adenylosuccinate lyase | 0.76 | 2.61 | 4.69 |
| | (ADSL) | | | |
| AICDA | activation-induced cytidine | 0.45 | 0.79 | 32.68 |
| | deaminase (AICDA) | | | |
| AKR1A1 | aldo-keto reductase family 1, | 1.14 | 2.24 | 3.17 |
| | member A1, aldehyde | | | |
| | reductase (AKR1A1) | | | |
| AKR1B1 | aldo-keto reductase family 1, | 1.11 | 2.21 | 5.10 |
| | member B1, aldose reductase, | | | |
| | (AKR1B1) | | | |
| ALDOA | aldolase A, fructose- | 1.06 | 1.80 | 4.26 |
| | bisphosphate (ALDOA) | | | |
| ALDOC | aldolase C, fructose- | 0.99 | 1.16 | 5.82 |
| | bisphosphate (ALDOC) | | | |
| AP2S1 | adaptor-related protein | 1.65 | 2.66 | 4.45 |
| | complex 2, sigma 1 subunit | | | |
| | (AP2S1) | | | |
| AP3S2 | adaptor-related protein | 1.46 | 1.98 | 3.01 |
| | complex 3, sigma 2 subunit | | | |
| | (AP3S2) | | | |
| APEX | APEX nuclease, | 1.07 | 2.55 | 3.93 |
| | multifunctional DNA repair | | | |
| | enzyme, (APEX) | | | |
| APRT | adenine | 0.99 | 1.82 | 4.09 |
| | phosphoribosyltransferase | | | |
| | (APRT) | | | |
| ARL2 | ADP-ribosylation factor-like 2 | 1.02 | 1.88 | 5.04 |
| | (ARL2) | | | |
| ARV1 | likely ortholog of yeast ARV1 | 0.98 | 1.57 | 4.41 |
| | (ARV1) | | | |
| ATIC | 5-aminoimidazole-4- | 1.06 | 2.42 | 4.19 |
| | carboxamide ribonucleotide | | | |
| | formyltransferase/IMP | | | |
| | cyclohydrolase (ATIC) | | | |
| ATOX1 | ATX1 antioxidant protein 1 | 1.13 | 2.44 | 5.67 |
| | homolog, yeast (ATOX1) | | | |
| ATP5D | ATP synthase, H⁺ transporting, | 0.93 | 1.27 | 4.40 |
| | mitochondrial F1 complex, | | | |
| | delta subunit (ATP5D) | | | |
| ATP5G1 | ATP synthase, H⁺ transporting, | 0.77 | 1.47 | 3.64 |
| | mitochondrial F0 complex, | | | |
| | subunit c, subunit 9, isoform 1 | | | |
| | (ATP5G1) | | | |
| ATP5H | ATP synthase, H⁺ transporting, | 1.20 | 2.97 | 4.96 |
| | mitochondrial F0 complex, | | | |
| | subunit d (ATP5H) | | | |
| ATP5J | ATP synthase, H⁺ transporting, | 1.15 | 2.32 | 3.45 |
| | mitochondrial F0 complex, | | | |
| | subunit F6 (ATP5J) | | | |
| ATP5J2 | ATP synthase, H⁺ transporting, | 1.44 | 2.41 | 10.53 |
| | mitochondrial F0 complex, | | | |
| | subunit f, isoform 2 (ATP5J2) | | | |
| B4GALT5 | UDP-Gal:betaGlcNAc beta | 0.87 | 3.19 | 4.35 |
| | 1,4-galactosyltransferase, | | | |
| | polypeptide 5 (B4GALT5) | | | |
| BATF | basic leucine zipper | 0.93 | 5.53 | 19.27 |
| | transcription factor (BATF) | | | |
| BAχ | BCL2-associated X protein | 1.57 | 2.20 | 3.02 |
| | (BAX) | | | |
| BCL2A1 | BCL2-related protein A1 | 1.98 | 5.43 | 3.07 |
| | (BCL2A1) | | | |
| BLNK | B-cell linker (BLNK) | 0.71 | 1.99 | 3.30 |
| BST2 | bone marrow stromal cell | 0.91 | 1.59 | 5.04 |
| | antigen 2 (BST2) | | | |
| BYSL | bystin-like (BYSL) | 0.91 | 3.07 | 3.47 |
| C1QBP | complement component 1, q | 1.01 | 1.87 | 3.02 |
| | subcomponent binding protein | | | |
| | (C1QBP) | | | |
| CABYR | fibrousheathin II (CABYR) | 1.16 | 3.40 | 9.41 |
| CBR1 | carbonyl reductase 1 (CBR1) | 0.70 | 1.80 | 5.17 |
| CCL17 | small inducible cytokine | 0.94 | 0.72 | 5.21 |
| | subfamily A [Cys-Cys], | | | |
| | member 17 (CCL17) | | | |
| CCL3 | small inducible cytokine A3 | 4.08 | 13.56 | 1.43 |
| | (CCL3) | | | |
| CCL4 | small inducible cytokine A4 | 3.54 | 16.22 | 2.26 |
| | (CCL4) | | | |
| CCL7 | small inducible cytokine A7 | 0.97 | 10.43 | 1.58 |
| | [monocyte chemotactic protein | | | |
| | 3] (CCL7) | | | |
| CD40 | tumor necrosis factor receptor | 0.79 | 3.28 | 3.05 |
| | superfamily, member 5 | | | |
| | (CD40) | | | |
| CD59 | CD59 antigen p18-20, antigen | 0.83 | 1.10 | 3.04 |
| | identified by monoclonal | | | |
| | antibodies 16.3A5, EJ16, EJ30, | | | |
| | EL32 and G344 (CD59) | | | |
| CDK4 | cyclin-dependent kinase 4 | 0.85 | 2.46 | 3.36 |
| | (CDK4) | | | |
| CETN2 | centrin, EF-hand protein, 2 | 0.90 | 1.46 | 3.10 |
| | (CETN2) | | | |
| CFL1 | cofilin 1, non-muscle (CFL1) | 0.82 | 1.20 | 4.13 |
| CFLAR | CASP8 and FADD-like | 1.05 | 3.22 | 7.21 |
| | apoptosis regulator (CFLAR) | | | |
| CLPB | suppressor of potassium | 0.71 | 1.31 | 3.26 |
| | transport defect 3 (CLPB) | | | |
| CLPP | ClpP caseinolytic protease, | 0.97 | 3.03 | 4.10 |
| | ATP-dependent, proteolytic | | | |
| | subunit homolog, E. coli | | | |
| | (CLPP) | | | |
| COX11 | COX11 homolog, cytochrome | 0.90 | 1.57 | 3.93 |
| | c oxidase assembly protein, | | | |
| | yeast (COX11) | | | |
| COX5A | cytochrome c oxidase subunit | 1.05 | 1.61 | 3.40 |
| | Va (COX5A) | | | |
| COX6C | cytochrome c oxidase subunit | 1.69 | 1.86 | 4.26 |
| | VIc (COX6C) | | | |
| COX7B | cytochrome c oxidase subunit | 1.34 | 2.37 | 3.17 |
| | VIIb (COX7B) | | | |
| CSK | c-src tyrosine kinase (CSK) | 0.77 | 1.27 | 3.49 |
| CSRP2 | cysteine and glycine-rich | 1.08 | 1.39 | 4.41 |
| | protein 2 (CSRP2) | | | |
| CTSH | cathepsin H (CTSH) | 1.00 | 2.76 | 5.46 |
| DAP13 | 13 kDa differentiation- | 1.14 | 2.06 | 3.71 |
| | associated protein (DAP13) | | | |
| DCPS | mRNA decapping enzyme | 1.36 | 1.55 | 4.16 |
| | (DCPS) | | | |
| DDX18 | DEAD/H, Asp-Glu-Ala- | 0.66 | 1.54 | 4.42 |
| | Asp/His box polypeptide 18 | | | |
| | [Myc-regulated] (DDX18) | | | |
| DDX37 | DEAD/H, Asp-Glu-Ala- | 1.34 | 2.12 | 3.48 |
| | Asp/His box polypeptide 37 | | | |
| | (DDX37) | | | |
| DEPC-1 | prostate cancer antigen-1 | 0.74 | 2.23 | 3.06 |
| | (DEPC-1) | | | |
| DHCR24 | 24-dehydrocholesterol | 0.87 | 0.94 | 12.43 |
| | reductase (DHCR24) | | | |
| DLEU1 | deleted in lymphocytic | 1.16 | 1.36 | 5.49 |
| | leukemia, 1 (DLEU1) | | | |
| DPM2 | dolichyl-phosphate | 0.76 | 2.49 | 3.72 |
| | mannosyltransferase | | | |
| | polypeptide 2, regulatory | | | |
| | subunit (DPM2) | | | |
| DPP3 | dipeptidylpeptidase III (DPP3) | 1.04 | 1.86 | 4.58 |
| DPP-I | prepro dipeptidyl peptidase I | 1.06 | 3.45 | 2.49 |
| | (DPP-I) (CTSC) | | | |
| DSCR2 | Down syndrome critical region | 0.90 | 3.29 | 4.61 |
| | gene 2 (DSCR2) | | | |
| DTX1 | deltex homolog 1, Drosophila | 0.51 | 0.80 | 3.80 |
| | (DTX1) | | | |
| DTYMK | deoxythymidylate | 1.05 | 2.07 | 4.96 |
| | kinase_thymidylate kinase | | | |
| | (DTYMK) | | | |
| EBI3 | Epstein-Barr virus induced | 0.95 | 4.77 | 16.43 |
| | gene 3, componente de IL35, | | | |
| | (EBI3) | | | |
| EBNA1BP2 | EBNA1 binding protein 2 | 0.85 | 3.03 | 3.90 |
| | (EBNA1BP2) | | | |
| EGF | epidermal growth factor [beta- | 1.41 | 1.68 | 3.59 |
| | urogastrone] (EGF) | | | |
| EIF2B3 | eukaryotic translation initiation | 0.81 | 2.83 | 5.07 |
| | factor 2B, subunit 3 [gamma, | | | |
| | 58 kD] (EIF2B3) | | | |
| EIF3C | eukaryotic translation initiation | 0.26 | 1.62 | 3.91 |
| | factor 3, subunit 8 [110 kD] | | | |
| | (EIF3C) | | | |
| EIF5A | eukaryotic translation initiation | 1.40 | 1.98 | 4.86 |
| | factor 5A (EIF5A) | | | |
| ENO1 | enolase 1, alpha (ENO1) | 0.82 | 2.82 | 4.98 |
| ETFA | electron-transfer-flavoprotein, | 0.77 | 1.86 | 3.91 |
| | alpha polypeptide [glutaric | | | |
| | aciduria II] (ETFA) | | | |
| FABP5 | fatty acid binding protein 5 | 1.22 | 4.06 | 8.41 |
| | [psoriasis-associated] (FABP5) | | | |
| FDPS | farnesyl diphosphate synthase | 0.82 | 1.07 | 3.05 |
| | [farnesyl pyrophosphate | | | |
| | synthetase, | | | |
| | dimethylallyltranstransferase, | | | |
| | geranyltranstransferase] | | | |
| | (FDPS) | | | |
| FMR2 | fragile X mental retardation 2 | 0.91 | 1.32 | 3.09 |
| | (FMR2) | | | |
| FRDA | Friedreich ataxia (FRDA) | 0.81 | 1.52 | 3.80 |
| GALE | galactose-4-epimerase, UDP- | 0.64 | 1.38 | 3.74 |
| | (GALE) | | | |
| GBGT1 | Forssman glycolipid synthetase | 2.16 | 2.30 | 5.52 |
| | [FS], mRNA (GBGT1) | | | |
| GBP1 | guanylate binding protein 1, | 0.96 | 1.42 | 4.35 |
| | interferon-inducible, 67 kD | | | |
| | (GBP1) | | | |
| GCN5L1 | GCN5 general control of | 1.10 | 1.74 | 5.35 |
| | amino-acid synthesis 5-like 1 | | | |
| | [yeast] (GCN5L1) | | | |
| GCSH | glycine cleavage system | 1.49 | 3.67 | 6.88 |
| | protein H [aminomethyl | | | |
| | carrier] (GCSH) | | | |
| GG2-1 | TNF-induced protein (GG2-1) | 0.98 | 2.32 | 3.55 |
| GNG10 | guanine nucleotide binding | 1.05 | 2.05 | 3.08 |
| | protein 10 (GNG10) | | | |
| GOT2 | glutamic-oxaloacetic | 0.58 | 2.12 | 4.58 |
| | transaminase 2, mitochondrial | | | |
| | (GOT2) | | | |
| GPS1 | G protein pathway suppressor | 1.52 | 1.18 | 4.03 |
| | 1 (GPS1) | | | |
| GRHPR | glyoxylate | 0.84 | 2.00 | 12.06 |
| | reductase/hydroxypyruvate | | | |
| | reductase (GRHPR) | | | |
| GRN | granulin (GRN) | 0.78 | 1.78 | 3.31 |
| GRSF1 | G-rich RNA sequence binding | 0.79 | 2.03 | 3.33 |
| | factor 1 (GRSF1) | | | |
| GSR | glutathione reductase (GSR) | 0.72 | 2.23 | 4.64 |
| GSTP1 | glutathione S-transferase pi | 1.04 | 3.50 | 9.52 |
| | (GSTP1) | | | |
| GTF2H4 | general transcription factor | 0.78 | 2.87 | 9.43 |
| | IIH, polypeptide 4 [52 kD | | | |
| | subunit] (GTF2H4) | | | |
| HCK | hemopoietic cell kinase (HCK) | 0.93 | 0.89 | 8.53 |
| HCLS1 | hematopoietic cell-specific | 0.54 | 1.41 | 3.06 |
| | Lyn substrate 1 (HCLS1) | | | |
| HIG2 | hypoxia-inducible protein 2 | 0.81 | 2.17 | 6.30 |
| | (HIG2) | | | |
| HKE2 | HLA class II region expressed | 0.84 | 2.12 | 3.49 |
| | gene KE2 (HKE2), mRNA | | | |
| | (PFDN6) | | | |
| HLA-DRBI | MHC class II DPw3-alpha-1 | 0.90 | 1.98 | 3.22 |
| | chain mRNA, complete cds | | | |
| | (HLA-DRBI) | | | |
| HNRPA2B1 | heterogeneous nuclear | 0.99 | 2.41 | 3.77 |
| | ribonucleoprotein A2/B1 | | | |
| | (HNRPA2B1) | | | |
| HSP90AA1 | heat shock 90 kD protein 1, | 0.64 | 2.07 | 3.03 |
| | alpha (HSP90AA1) | | | |
| HSP90AB1 | heat shock 90 kD protein 1, | 0.61 | 1.99 | 3.90 |
| | beta (HSP90AB1) | | | |
| HSPB1 | heat shock 27 kD protein 1 | 1.01 | 1.50 | 3.21 |
| | (HSPB1) | | | |
| HSPC051 | ubiquinol-cytochrome c | 1.08 | 1.95 | 3.08 |
| | reductase complex [7.2 Kd] | | | |
| | (HSPC051) | | | |
| ICT1 | immature colon carcinoma | 1.42 | 1.62 | 3.07 |
| | transcript 1 (ICT1) | | | |
| IL10 | interleukin 10 (IL10) | 1.36 | 4.87 | 1.84 |
| IL2RA | interleukin 2 receptor, alpha | 1.28 | 6.71 | 8.71 |
| | [CD25] (IL2RA) | | | |
| IL6 | interleukin 6 (IL6) | 1.78 | 5.79 | 2.49 |
| IL8 | interleukin 8 (IL8) | 1.23 | 2.47 | 3.14 |
| IMPDH2 | IMP [inosine monophosphate] | 0.85 | 2.00 | 5.96 |
| | dehydrogenase 2 (IMPDH2) | | | |
| ITM3 | integral membrane protein 3 | 1.31 | 1.87 | 4.22 |
| | (ITM3) | | | |
| KHDRBS1 | GAP-associated tyrosine | 0.91 | 1.33 | 3.14 |
| | phosphoprotein p62 [Sam68] | | | |
| | (KHDRBS1) | | | |
| LCAT | lecithin-cholesterol | 0.69 | 2.81 | 5.27 |
| | acyltransferase (LCAT) | | | |
| LDHB | lactate dehydrogenase B | 0.63 | 1.39 | 4.85 |
| | (LDHB) | | | |
| LEP16 | epidermal differentiation | 1.87 | 2.84 | 3.30 |
| | complex protein like protein | | | |
| | (LEP16) | | | |
| LGALS3 | lectin, galactoside-binding, | 0.82 | 2.15 | 7.03 |
| | soluble, 3 [galectin 3] | | | |
| | (LGALS3) | | | |
| LILRB2 | leukocyte immunoglobulin- | 1.08 | 2.28 | 3.04 |
| | like receptor, subfamily B | | | |
| | [with TM and ITIM domains], | | | |
| | member 2 (LILRB2) | | | |
| LSM2 | U6 snRNA-associated Sm-like | 1.13 | 2.08 | 4.33 |
| | protein (LSM2) | | | |
| LTA | lymphotoxin alpha [TNF | 1.95 | 5.08 | 1.09 |
| | superfamily, member 1] (LTA) | | | |
| MAAT1 | melanoma-associated antigen | 0.92 | 2.30 | 3.91 |
| | recognized by cytotoxic T | | | |
| | lymphocytes (MAAT1) | | | |
| MAP4 | microtubule-associated protein | 1.12 | 2.04 | 3.53 |
| | 4 (MAP4) | | | |
| MBTPS1 | membrane-bound transcription | 0.61 | 3.70 | 0.56 |
| | factor protease, site 1 | | | |
| | (MBTPS1) | | | |
| MDH2 | malate dehydrogenase 2, NAD | 1.00 | 1.50 | 3.10 |
| | [mitochondrial] (MDH2) | | | |
| MEA | male-enhanced antigen (MEA) | 0.96 | 1.33 | 3.39 |
| MEF2B | MADS box transcription | 4.18 | 5.51 | 2.26 |
| | enhancer factor 2, polypeptide | | | |
| | B [myocyte enhancer factor | | | |
| | 2B] (MEF2B) | | | |
| MEP50 | MEP50 protein (MEP50) | 0.80 | 1.99 | 3.61 |
| MMP7 | matrix metalloproteinase 7 | 0.97 | 5.04 | 0.35 |
| | [matrilysin, uterine] (MMP7) | | | |
| MPI | mannose phosphate isomerase | 1.43 | 2.56 | 5.11 |
| | (MPI) | | | |
| MRPL11 | Mitochondrial Ribosomal | 1.56 | 2.65 | 8.65 |
| | Protein L11 (MRPL11) | | | |
| MRPL11 | mitochondrial ribosomal | 1.25 | 1.85 | 5.03 |
| | protein L11 (MRPL11) | | | |
| MRPL12 | mitochondrial ribosomal | 1.06 | 3.63 | 6.01 |
| | protein L12 (MRPL12) | | | |
| MRPL23 | mitochondrial ribosomal | 1.10 | 2.00 | 4.70 |
| | protein L23 (MRPL23) | | | |
| MRPL24 | mitochondrial ribosomal | 0.72 | 1.78 | 4.30 |
| | protein L24 (MRPL24) | | | |
| MRPL3 | mitochondrial ribosomal | 0.71 | 1.89 | 3.12 |
| | protein L3 (MRPL3) | | | |
| MRPL4 | mitochondrial ribosomal | 0.86 | 1.87 | 3.49 |
| | protein L4 (MRPL4) | | | |
| MRPL45 | mitochondrial ribosomal | 1.02 | 1.99 | 3.11 |
| | protein L45 (MRPL45) | | | |
| MBPS11 | mitochondrial ribosomal | 1.12 | 4.02 | 6.11 |
| | protein S11 (MRPS11) | | | |
| MBPS12 | mitochondrial ribosomal | 1.50 | 3.69 | 3.59 |
| | protein S12 (MRPS12) | | | |
| MBPS15 | mitochondrial ribosomal | 0.94 | 1.97 | 3.37 |
| | protein S15 (MRPS15) | | | |
| MBPS16 | mitochondrial ribosomal | 1.06 | 2.37 | 4.05 |
| | protein S16 (MRPS16) | | | |
| MRPS24 | mitochondrial ribosomal | 0.95 | 1.26 | 3.35 |
| | protein S24 (MRPS24) | | | |
| MRPS25 | mitochondrial ribosomal | 1.17 | 1.40 | 3.51 |
| | protein S25 (MRPS25) | | | |
| MRPS28 | mitochondrial ribosomal | 0.95 | 2.41 | 5.08 |
| | protein S28 (MRPS28) | | | |
| MRPS31 | mitochondrial ribosomal | 0.86 | 2.36 | 3.90 |
| | protein S31 (MRPS31) | | | |
| MTHFD1 | methylenetetrahydrofolate | 0.85 | 0.93 | 3.73 |
| | dehydrogenase [NADP⁺ | | | |
| | dependent] (MTHFD1) | | | |
| MUC1 | Human polymorphic epithelial | 0.47 | 0.56 | 5.79 |
| | mucin (PEM) (MUC1) | | | |
| MYL6B | myosin light chain 1 slow a | 1.39 | 0.90 | 5.24 |
| | (MYL6B) | | | |
| NAA10 | ARD1 homolog, N- | 1.08 | 1.88 | 3.71 |
| | acetyltransferase, S. cerevisiae, | | | |
| | (NAA10) | | | |
| NBAS | neuroblastoma-amplified | 0.86 | 1.67 | 3.15 |
| | protein (NBAS) | | | |
| NCF2 | neutrophil cytosolic factor 2 | 0.74 | 0.99 | 17.97 |
| | [65 kD, chronic granulomatous | | | |
| | disease, autosomal 2] (NCF2) | | | |
| NCL | nucleolin (NCL) | 0.82 | 2.29 | 3.83 |
| NDUFA2 | NADH dehydrogenase | 1.53 | 2.39 | 4.14 |
| | [ubiquinone] 1 alpha | | | |
| | subcomplex, 2 [8 kD, B8] | | | |
| | (NDUFA2) | | | |
| NDUFA3 | NADH dehydrogenase | 1.59 | 2.04 | 3.07 |
| | [ubiquinone] 1 alpha | | | |
| | subcomplex, 3 [9 kD, B9] | | | |
| | (NDUFA3) | | | |
| NDUFA4 | NADH dehydrogenase | 1.34 | 1.73 | 3.86 |
| | [ubiquinone] 1 alpha | | | |
| | subcomplex, 4 [9 kD, MLRQ] | | | |
| | (NDUFA4) | | | |
| NDUFB1 | NADH dehydrogenase | 1.58 | 1.94 | 4.24 |
| | [ubiquinone] 1 beta | | | |
| | subcomplex, 1 [7 kD, MNLL] | | | |
| | (NDUFB1) | | | |
| NDUFB10 | NADH dehydrogenase | 1.42 | 1.84 | 3.18 |
| | [ubiquinone] 1 beta | | | |
| | subcomplex, 10 [22 kD, PDSW] | | | |
| | (NDUFB10) | | | |
| NDUFB6 | NADH dehydrogenase | 1.08 | 1.50 | 3.82 |
| | [ubiquinone] 1 beta | | | |
| | subcomplex, 6 [17 kD, B17] | | | |
| | (NDUFB6) | | | |
| NDUFB8 | NADH dehydrogenase | 1.07 | 2.11 | 3.89 |
| | [ubiquinone] 1 beta | | | |
| | subcomplex, 8 [19 kD, ASHI] | | | |
| | (NDUFB8) | | | |
| NDUFB9 | NADH dehydrogenase | 1.15 | 2.60 | 3.49 |
| | [ubiquinone] 1 beta | | | |
| | subcomplex, 9 [22 kD, B22] | | | |
| | (NDUFB9) | | | |
| NDUFC1 | NADH dehydrogenase | 1.16 | 1.72 | 3.47 |
| | [ubiquinone] 1 (NDUFC1) | | | |
| NDUFS8 | NADH dehydrogenase | 1.62 | 2.16 | 6.54 |
| | [ubiquinone] Fe—S protein 8 | | | |
| | [23 kD, NADH-coenzyme Q | | | |
| | reductase] (NDUFS8) | | | |
| NEDD8 | neural precursor cell | 1.09 | 1.97 | 4.28 |
| | expressed, developmentally | | | |
| | down-regulated 8 (NEDD8) | | | |
| NFYB | nuclear transcription factor Y, | 1.08 | 1.72 | 3.28 |
| | beta (NFYB) | | | |
| NME1 | non-metastatic cells 1, protein | 1.40 | 6.48 | 9.22 |
| | (NM23A) expressed in | | | |
| | (NME1) | | | |
| NME2 | non-metastatic cells 2, | 0.99 | 1.29 | 3.68 |
| | (NME2) | | | |
| NTHL1 | nth endonuclease III-like 1 [E. | 0.92 | 0.99 | 5.37 |
| | coli] (NTHL1) | | | |
| NUDT1 | nudix [nucleoside diphosphate | 0.84 | 1.19 | 3.15 |
| | linked moiety X]-type motif 1 | | | |
| | (NUDT1) | | | |
| PA2G4 | proliferation-associated 2G4, | 0.77 | 2.05 | 3.68 |
| | 38 kD (PA2G4) | | | |
| PAFAH1B3 | platelet-activating factor | 1.09 | 1.26 | 4.90 |
| | acetylhydrolase, isoform Ib, | | | |
| | gamma subunit [29 kD] | | | |
| | (PAFAH1B3) | | | |
| PAICS | phosphoribosylaminoimidazole | 0.94 | 2.64 | 4.28 |
| | carboxylase, | | | |
| | phosphoribosylaminoimidazole | | | |
| | succinocarboxamide | | | |
| | synthetase (PAICS) | | | |
| PAICS | phosphoribosylaminoimidazole | 0.87 | 2.49 | 4.25 |
| | carboxylase (PAICS) | | | |
| PAM16 | Mitochondrial Presequence | 1.06 | 2.53 | 4.16 |
| | Translocase Associated Motor | | | |
| | 16 (PAM16) | | | |
| PANK | pantothenate kinase (PANK) | 0.98 | 1.45 | 3.39 |
| PBP | prostatic binding protein (PBP) | 0.88 | 1.57 | 3.07 |
| PCBD1 | Pterin-4 Alpha-Carbinolamine | 1.16 | 1.78 | 13.76 |
| | Dehydratase 1 (PCBD1) | | | |
| PDAP1 | PDGFA associated protein 1 | 0.77 | 1.88 | 3.27 |
| | (PDAP1) | | | |
| PDCD5 | programmed cell death 5 | 1.11 | 1.90 | 4.00 |
| | (PDCD5) | | | |
| PEPD | peptidase D (PEPD) | 1.32 | 2.30 | 6.28 |
| PET112L | PET112-like [yeast] | 0.71 | 2.39 | 3.71 |
| | (PET112L) | | | |
| PI3K sub 110 | Human phosphatidylinositol 3- | 0.89 | 1.24 | 3.20 |
| | kinase catalytic subunit | | | |
| | p110delta mRNA, complete | | | |
| | cds (PI3K sub 110) | | | |
| PKM | pyruvate kinase, muscle | 0.96 | 1.97 | 15.51 |
| | (PKM) | | | |
| PLEK | pleckstrin (PLEK) | 1.76 | 3.54 | 2.89 |
| POLA | polymerase [DNA directed], | 1.49 | 1.03 | 3.83 |
| | alpha (POLA) | | | |
| POLA2 | polymerase [DNA directed], | 1.02 | 1.69 | 3.03 |
| | alpha [70 kD] (POLA2) | | | |
| POLE4 | polymerase [DNA directed], | 1.11 | 1.01 | 3.18 |
| | epsilon 4 [p12 subunit] | | | |
| | (POLE4) | | | |
| POLR2F | polymerase [RNA] II [DNA | 1.42 | 1.88 | 3.18 |
| | directed] polypeptide F | | | |
| | (POLR2F) | | | |
| POLR2H | polymerase [RNA] II [DNA | 0.86 | 2.28 | 4.05 |
| | directed] polypeptide H | | | |
| | (POLR2H) | | | |
| POLR2L | polymerase [RNA] II [DNA | 1.14 | 2.39 | 3.86 |
| | directed] polypeptide L | | | |
| | [7.6 kD] (POLR2L) | | | |
| POP5 | RNase MRP/RNase P protein- | 0.99 | 1.69 | 3.37 |
| | like (POP5) | | | |
| POU2AF1 | POU domain, class 2, | 1.69 | 2.03 | 6.10 |
| | associating factor 1 | | | |
| | (POU2AF1) | | | |
| PP15 | nuclear transport factor 2 | 1.11 | 2.38 | 6.08 |
| | [placental protein 15] (PP15) | | | |
| PP3111 | PP3111 protein (PP3111) | 0.81 | 1.71 | 3.32 |
| PPIE | peptidylprolyl isomerase E | 0.77 | 2.31 | 3.31 |
| | [cyclophilin E] (PPIE) | | | |
| PPP1CA | protein phosphatase 1, catalytic | 0.90 | 1.88 | 4.26 |
| | subunit, alpha isoform | | | |
| | (PPP1CA) | | | |
| PPP1R7 | protein phosphatase 1, | 2.02 | 1.89 | 3.35 |
| | regulatory subunit 7 (PPP1R7) | | | |
| PRDX1 | peroxiredoxin 1 (PRDX1) | 0.77 | 2.20 | 3.65 |
| PRMT1 | HMT1 hnRNP | 0.59 | 1.85 | 3.57 |
| | methyltransferase-like 2 | | | |
| | (PRMT1) | | | |
| PSMB5 | proteasome[prosome, | 0.86 | 2.28 | 3.30 |
| | macropain]subunit, beta type, | | | |
| | 5 (PSMB5) | | | |
| PSMD1 | proteasome[prosome, | 1.30 | 3.05 | 3.99 |
| | macropain]26S subunit, non- | | | |
| | ATPase, 1 (PSMD1) | | | |
| PSMD4 | proteasome[prosome, | 0.76 | 2.48 | 3.60 |
| | macropain]26S subunit, non- | | | |
| | ATPase, 4 (PSMD4) | | | |
| PSME1 | proteasome[prosome, | 0.98 | 1.41 | 3.05 |
| | macropain]activator subunit 1 | | | |
| | [PA28 alpha] (PSME1) | | | |
| PSME2 | proteasome[prosome, | 0.82 | 2.59 | 4.91 |
| | macropain]activator subunit 2 | | | |
| | [PA28 beta] (PSME2) | | | |
| PTGES2 | prostaglandin E synthase 2 | 1.06 | 1.83 | 4.52 |
| | (PTGES2) | | | |
| PYCR1 | pyrroline-5-carboxylate | 0.77 | 4.74 | 7.43 |
| | reductase 1 (PYCR1) | | | |
| RAB13 | RAB13, member RAS | 1.19 | 1.89 | 17.99 |
| | oncogene family (RAB13) | | | |
| RAB34 | RAB34, member RAS | 0.98 | 1.48 | 4.13 |
| | oncogene family (RAB34) | | | |
| RAB34 | RAB34, member RAS | 0.90 | 1.70 | 4.04 |
| | oncogene family (RAB34) | | | |
| RAB9P40 | Rab9 effector p40 (RAB9P40) | 1.27 | 2.99 | 4.68 |
| RANBP1 | RAN binding protein 1 | 0.95 | 3.18 | 4.83 |
| | (RANBP1) | | | |
| REC14 | recombination protein REC14 | 0.97 | 1.78 | 3.63 |
| | (REC14) | | | |
| RFC4 | replication factor C [activator | 0.74 | 1.99 | 4.06 |
| | 1] 4 [37 kD] (RFC4) | | | |
| RIDA | translational inhibitor protein | 1.39 | 2.93 | 5.48 |
| | p14.5 (HRSP12) (RIDA) | | | |
| RPL26L1 | ribosomal protein L26-like 1 | 1.26 | 2.85 | 7.13 |
| | (RPL26L1) | | | |
| RPS26 | ribosomal protein S26 (RPS26) | 1.22 | 1.25 | 3.11 |
| RSU1 | Ras suppressor protein 1 | 0.77 | 1.39 | 3.38 |
| | (RSU1) | | | |
| RUVBL2 | RuvB-like 2 [E. coli] | 0.74 | 1.29 | 3.19 |
| | (RUVBL2) | | | |
| S100A4 | S100 calcium binding protein | 1.16 | 1.02 | 4.10 |
| | A4 [calcium protein, | | | |
| | calvasculin, metastasin, murine | | | |
| | placental homolog] (S100A4) | | | |
| SCAMP3 | secretory carrier membrane | 0.87 | 2.67 | 3.77 |
| | protein 3 (SCAMP3) | | | |
| SDF2L1 | stromal cell-derived factor 2- | 1.04 | 1.92 | 5.97 |
| | like 1 (SDF2L1) | | | |
| SEC61B | protein translocation complex | 0.91 | 1.30 | 3.53 |
| | beta (SEC61B) | | | |
| SEPX1 | selenoprotein X, 1 (SEPX1) | 1.00 | 1.92 | 5.24 |
| SET | SET translocation [myeloid | 0.93 | 1.43 | 3.51 |
| | leukemia-associated] (SET) | | | |
| SFRS9 | splicing factor, arginine/serine- | 0.93 | 1.80 | 3.39 |
| | rich 9 (SFRS9) | | | |
| SHMT2 | serine | 0.86 | 2.36 | 4.76 |
| | hydroxymethyltransferase 2 | | | |
| | [mitochondrial] (SHMT2) | | | |
| SIP | Siah-interacting protein (SIP) | 0.88 | 1.41 | 3.50 |
| SLC23A2 | solute carrier family 23 | 1.02 | 1.44 | 3.58 |
| | [nucleobase transporters], | | | |
| | member 2 (SLC23A2) | | | |
| SLC38A5 | solute carrier family 38, | 1.33 | 2.17 | 6.77 |
| | member 5 (SLC38A5) | | | |
| SMS | spermine synthase (SMS) | 0.83 | 1.71 | 3.27 |
| SNAP23 | synaptosomal-associated | 6.25 | 5.85 | 3.63 |
| | protein, 23 kD (SNAP23) | | | |
| SNRPA | small nuclear | 0.77 | 0.99 | 3.22 |
| | ribonucleoprotein polypeptide | | | |
| | A (SNRPA) | | | |
| SNRPD3 | small nuclear | 1.51 | 2.48 | 3.71 |
| | ribonucleoprotein D3 | | | |
| | polypeptide [18 kD] (SNRPD3) | | | |
| SNRPF | small nuclear | 1.04 | 1.88 | 3.71 |
| | ribonucleoprotein polypeptide | | | |
| | F (SNRPF) | | | |
| SOD1 | superoxide dismutase 1, | 1.06 | 1.68 | 3.14 |
| | soluble [amyotrophic lateral | | | |
| | sclerosis 1 [adult]] (SOD1) | | | |
| SPS2 | selenophosphate synthetase 2 | 0.74 | 0.91 | 3.49 |
| | (SPS2) | | | |
| SRM | spermidine synthase (SRM) | 0.80 | 3.02 | 4.15 |
| SSRP1 | structure specific recognition | 0.55 | 1.43 | 3.71 |
| | protein 1 (SSRP1) | | | |
| STAT5A | signal transducer and activator | 1.00 | 2.69 | 3.38 |
| | of transcription 5A (STAT5A) | | | |
| STEAP | six transmembrane epithelial | 0.73 | 1.80 | 6.30 |
| | antigen of the prostate | | | |
| | (STEAP) | | | |
| STOML2 | stomatin (EPB72)-like 2 | 0.89 | 2.24 | 4.27 |
| | (STOML2) | | | |
| STX8 | syntaxin 8 (STX8) | 1.01 | 0.87 | 3.20 |
| TALDO1 | transaldolase 1 (TALDO1) | 0.58 | 2.40 | 8.88 |
| TCF1 | 6-pyruvoyl-tetrahydropterin | 0.96 | 1.60 | 13.08 |
| | synthase/dimerization cofactor | | | |
| | of hepatocyte nuclear factor 1 | | | |
| | alpha (TCF1)(PCBD) | | | |
| TCFL5 | transcription factor-like 5 | 0.83 | 2.13 | 4.72 |
| | [basic helix-loop-helix] | | | |
| | (TCFL5) | | | |
| THOP1 | thimet oligopeptidase 1 | 1.10 | 1.80 | 3.96 |
| | (THOP1) | | | |
| TIMM13 | translocase of inner | 0.96 | 2.10 | 3.75 |
| | mitochondrial membrane 13 | | | |
| | homolog [yeast] (TIMM13) | | | |
| TIMM8B | translocase of inner | 1.16 | 2.54 | 3.47 |
| | mitochondrial membrane 8 | | | |
| | homolog B [yeast] (TIMM8B) | | | |
| TMEM4 | transmembrane protein 4 | 1.25 | 2.70 | 4.20 |
| TNFRSF13B | transmembrane activator and | 0.95 | 1.23 | 3.65 |
| | CAML interactor [TACI], | | | |
| | mRNA.(TNFRSF13B) | | | |
| TNFRSF18 | tumor necrosis factor receptor | 1.46 | 3.24 | 2.03 |
| | superfamily, member 18 | | | |
| | (TNFRSF18) | | | |
| TOMM22 | translocase of outer | 1.03 | 2.26 | 3.60 |
| | mitochondrial membrane 22 | | | |
| | homolog [yeast] (TOMM22) | | | |
| TP53 | tumor protein p53]Li-Fraumni | 0.98 | 1.91 | 4.11 |
| | syndrome] (TP53) | | | |
| TPI1 | triosephosphate isomerase 1 | 0.95 | 2.46 | 5.56 |
| | (TPI1) | | | |
| TRAF1 | TNF receptor-associated factor | 1.81 | 8.48 | 5.90 |
| | 1 (TRAF1) | | | |
| TRC8 | Human multiple membrane | 1.08 | 4.98 | 2.14 |
| | spanning receptor TRC8 | | | |
| | (TRC8) | | | |
| TRIP10 | thyroid hormone receptor | 1.46 | 3.05 | 11.29 |
| | interactor 10 (TRIP10) | | | |
| TTC1 | tetratricopeptide repeat domain | 0.93 | 2.22 | 3.08 |
| | 1 (TTC1) | | | |
| TUBA6 | tubulin alpha 6 (TUBA6) | 0.81 | 1.23 | 3.69 |
| TXN | thioredoxin (TXN) | 1.18 | 3.74 | 13.62 |
| TXNRD1 | thioredoxin reductase 1 | 0.80 | 3.23 | 3.94 |
| | (TXNRD1) | | | |
| UBL5 | ubiquitin-like 5 (UBL5) | 1.52 | 1.83 | 3.41 |
| Ubls | mRNA for ubiquitin-like | 0.94 | 1.42 | 3.12 |
| | protein, complete cds (Ubls) | | | |
| UQCRB | low molecular mass | 1.27 | 2.78 | 3.53 |
| | ubiquinone-binding protein | | | |
| | [9.5 kD] [QP-C], | | | |
| | mRNA.(UQCRB) | | | |
| USP5 | ubiquitin specific protease 5 | 0.52 | 1.42 | 6.30 |
| | [isopeptidase T] (USP5) | | | |
| VARS2 | valyl-tRNA synthetase 2 | 1.12 | 2.44 | 4.73 |
| | (VARS2) | | | |
| VTI2 | vesicle-associated soluble NSF | 0.97 | 2.15 | 3.27 |
| | attachment protein receptor | | | |
| | (VTI2), mRNA. (VTI1B) | | | |
| WBSCR18 | Williams Beuren syndrome | 1.33 | 1.56 | 3.01 |
| | chromosome region 18 | | | |
| | (WBSCR18) | | | |
| WNT8B | wingless-type MMTV | 0.90 | 1.56 | 3.20 |
| | integration site family, member | | | |
| | 8B (WNT8B) | | | |

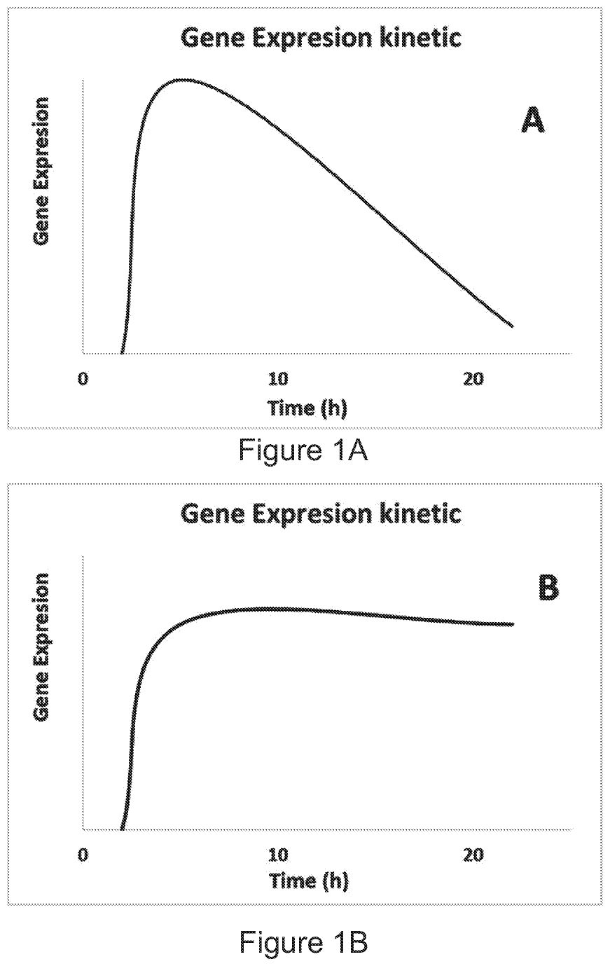

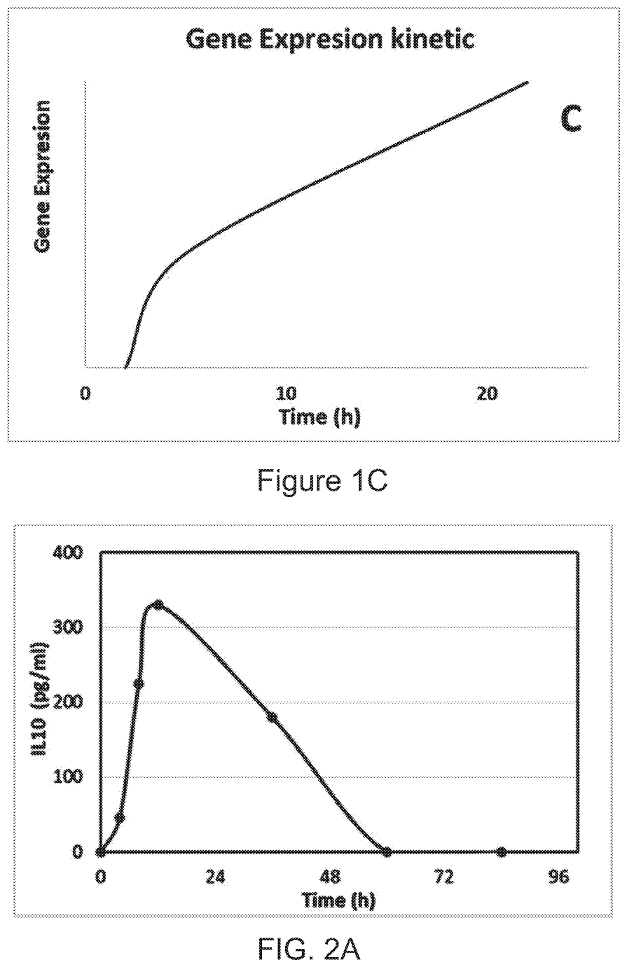

Kinetics of the mRNAs induction was not uniform for all genes, and roughly, three large groups were distinguished (FIGS. 1A-1C). FIG. 1A represents a group of genes induced at an early stage during incubation that reach a maximum level somewhere between 4 and 22 h, and then decaying to almost their initial level by the 22 h incubation time. These were named, “early genes” and they are mainly genes which codify for pro-inflammatory secreted proteins (Table 2).

TABLE 2
IMT504 early induce genes codifying secreted proteins

| Gene | Protein | Effects |
|---|---|---|
| CCL3 | C-C Motif Chemokine Ligand 3 | Induces chemotactic |
| | | mobilization of monocyte- |
| | | lineage cells and |
| | | lymphocytes into |
| | | inflammatory tissues. Also |
| | | regulates proliferation of |
| | | hematopoietic |
| | | stem/progenitor cells in the |
| | | bone marrow |
| CCL4 | C-C Motif Chemokine Ligand 4 | CC chemokine with |
| | | specificity for CCR5 |
| | | receptors. It is a |
| | | chemoattractant for natural |
| | | killer cells, monocytes and a |
| | | variety of other immune |
| | | cells. |
| CCL7 | C-C Motif Chemokine Ligand 7 | Chemotactic factor that |
| | | attracts monocytes, |
| | | eosinophils and MSC |
| IL-6 | Interleukin 6 | Essential for the final |
| | | differentiation of B-cells into |
| | | Ig-secreting cells. Involved |
| | | in lymphocyte and monocyte |
| | | differentiation. Required for |
| | | the generation of T_{H}17 cells |
| IL-10 | Interleukin 10 | Anti-inflammatory. Down- |
| | | regulates the expression of |
| | | T_{H}1 cytokine and |
| | | costimulatory molecules on |
| | | macrophages. Enhances B |
| | | cell survival, proliferation, |
| | | and antibody production. |
| | | This cytokine can block NF- |
| | | kappa B activity |
| LTA | Lymphotoxin alpha | Development and |
| | | maintenance of secondary |
| | | lymphoid tissues. LT also |
| | | plays an important role in |
| | | maintenance of lipid |
| | | homeostasis and liver |
| | | regeneration |

These proteins define a well know B cell phenotype which is commonly called “activated” B cells (58,59). Early genes are mainly targets of the NFκB1 transcription factor. The second group, represented by genes that codify cell surface proteins (Table 3), at difference of the genes included in the first group, reach a maximum level of mRNA concentration at an early time and this level is roughly maintained at least until the 22 h incubation time (FIG. 1B). These were named “early-late” genes.

TABLE 3
IMT504 early-late induced genes codifying cell surface proteins

| Gene | Protein | Effects |
|---|---|---|
| CD40 | CD40 surface antigen | Essential for T cell- |
| | | dependent IgG class |
| | | switching, memory B cell |
| | | development, and germinal |
| | | center formation |
| IL2Rα | Interleukin 2 Receptor | Regulates immune tolerance |
| | Subunit Alpha | by controlling regulatory T |
| | | cells (T_{REG}) activity. T_{REG} |
| | | cells suppress activation and |
| | | expansion of autoreactive T- |
| | | cells |
| LILRB2 | Leukocyte Immunoglobulin | Involved in the down- |
| | Like Receptor B2 | regulation of the immune |
| | | response and the |
| | | development of tolerance |

The third group, by far the most abundant, are represent by both, genes codifying secreted proteins (Table 4) and genes codifying cell surface proteins (Table 5). These were named “late genes”. These genes are mainly targets of the MYC, CREB and NRF1/2 transcription factors.

TABLE 4
IMT504 late induced genes codifying secreted proteins

| Gene | Protein | Effects |
|---|---|---|
| NENF | Neudesin | Neurotrophic Factor |
| GRN | Granulin Precursor | Mitogenic, Neurotrophic, |
| | | Neuroprotecting, Wound |
| | | Repairing and Anti- |
| | | inflammatory Factor |
| LGALS3 | Galectin 3 | Multifaceted regulation of |
| | | the Innate Immune |
| | | Response. Induce survival, |
| | | expansion, migration, and |
| | | immunomodulatory actions |
| | | of Mesenchymal Stem Cells |
| | | and Hepatic Progenitor Cells |
| EGF | Epidermal growth factor | Acts as a mitogenic factor |
| | | that plays a role in growth, |
| | | proliferation and |
| | | differentiation of numerous |
| | | cell types. Powerful |
| | | regulator of Stem Cells of |
| | | different tissues. |
| WNT8B | Wnt Family Member 8B | Signals trough the Canonical |
| | | Wnt Signaling pathway that |
| | | stimulate proliferation and |
| | | maintain stemness of diverse |
| | | adult Stem Cells |
| EBI3 | Epstein-Barr Virus Induced 3 | Subunit of two, |
| | | immunosuppressant and |
| | | anti-inflammatory |
| | | heterodimeric cytokines: IL- |
| | | 27 and IL-35 |
| APEX1 | Apurinic/Apyrimidinic | Central role in the cellular |
| | Endodeoxyribonuclease 1 | response to oxidative stress. |
| | | Secreted APEX1 inhibits |
| | | TNF-α-stimulated |
| | | Inflammation |

Products of the genes listed in Table 4 are almost exclusively anti-inflammatory and/or pro-tissue/organ reparatory proteins.

TABLE 5
IMT504 late induced genes codifying cell surface proteins

| Gene | Protein | Effects |
|---|---|---|
| MUC1 (PEM) | Mucin 1 | Inhibits activation of the |
| Inflammasome | | NLRP3 |
| HLA-DRB1 | Major histocompatibility | Mediates antigen |
| | antigen class II | presentation |
| MUC1 (PEM) | Mucin 1 | Inhibits activation of the |
| | | NLRP3 Inflammasome |
| HLA-DRB1 | Major histocompatibility | Mediates antigen |
| | antigen class II | presentation |
| CD59 | CD59 Surface Molecule | Potent inhibitor of the |
| | | complement membrane |
| | | attack complex |
| TNFRSF13B | TACI | Inhibits B cell expansion |
| | | and promotes differentiation |
| | | and survival of plasma cells. |
| | | Have a role in somatic |
| | | hypermutation and antibody |
| | | class switching. |

Products of the genes listed in Table 5 are involved in cell protection (MUC1 and CD59), antigen presentation (HLA-DR) and regulation of humoral immunity and autoimmune diseases (TAC1). Within the late expressed genes it is worth mentioning PTGES2 that codes for the enzyme prostaglandin E2 synthase. The product of this enzyme, prostaglandin E2, is a powerful regulator of the immune response (60).

Table 6 shows late genes induce by IMT504 codifying mitochondrial proteins. The extensive induction of these genes indicate mitochondriogenesis.

TABLE 6
IMT504 late induced genes codifying mitochondrial proteins

| Gene | Protein |
|---|---|
| TXN | Thioredoxin |
| PYCR1 | Pyrroline-5-Carboxylate Reductase 1 |
| MRPL12 | mitochondrial ribosomal protein L12 |
| VARS2 | valyl-tRNA Synthetase 2 |
| GOT2 | Glutamic-Oxaloacetic Transaminase 2 |
| NDUFB6 | NADH:Ubiquinone Oxidoreductase Subunit B6 |
| RUVBL2 | RuvB Like AAA ATPase 2 |
| MRPL23 | mitochondrial ribosomal protein L23 |
| NDUFC1 | NADH:Ubiquinone Oxidoreductase Subunit C1 |
| NDUFA4 | Cytochrome c oxidase subunit |
| NDUFB1 | NADH:Ubiquinone Oxidoreductase Subunit B1 |
| MRPS31 | Mitochondrial Ribosomal Protein S31 |
| TIMM13 | Translocase Of Inner Mitochondrial Membrane 13 |
| MRPL24 | Mitochondrial Ribosomal Protein L24 |
| MRPL11 | Mitochondrial Ribosomal Protein L11 |
| MRPL3 | Mitochondrial Ribosomal Protein L3 |
| NDUFS8 | NADH:Ubiquinone Oxidoreductase Core Subunit S8 |
| COX11 | Cytochrome C Oxidase Copper Chaperone |
| MDH2 | Malate Dehydrogenase 2 |
| NDUFA2 | NADH:Ubiquinone Oxidoreductase Subunit A2 |
| UQCRB | Ubiquinol-Cytochrome C Reductase Binding Protein |
| GRSF1 | G-Rich RNA Binding Factor 1 |
| MRPL11 | Mitochondrial Ribosomal Protein L11 |
| MRPS25 | Mitochondrial Ribosomal Protein S25 |
| T1MM8B | Translocase of Inner Mitochondrial Membrane 8A |
| STOML2 | Stomatin Like 2 |
| TOMM22 | Translocase of Outer Mitochondrial Membrane 22 |
| C1QBP | Complement C1q Binding Protein |
| MRPL4 | Mitochondrial Ribosomal Protein L4 |
| ATP5D | ATP Synthase, Mitochondrial F1 Complex, Delta |
| | Subunit |
| MRPL45 | Mitochondrial Ribosomal Protein L45 |
| MRPS24 | Mitochondrial Ribosomal Protein S24 |
| CLPP | Caseinolytic Mitochondrial Matrix Peptidase |
| | Proteolytic Subunit |
| ATP5G1 | ATP Synthase |
| COX5A | Cytochrome C Oxidase |
| ATP5J2 | ATP Synthase subunit F6 |
| NDUFA3 | NADH:Ubiquinone Oxidoreductase Subunit A3 |
| NDUFB8 | NADH:Ubiquinone Oxidoreductase Subunit B8 |
| SHMT2 | Serine Hydroxymethyltransferase 2 |
| MRPS16 | Mitochondrial Ribosomal Protein S16 |
| NDUFB10 | NADH:Ubiquinone Oxidoreductase Subunit B10 |
| MRPS12 | Mitochondrial Ribosomal Protein S12 |
| MRPS11 | Mitochondrial Ribosomal Protein S11 |

Table 7 shows IMT504 induced genes codifying extracellular vesicle associated proteins. Extracellular vesicle categories include large vesicles (more than 200 nm) and exosomes (less than 200 nm). Large extracellular vesicles could contain complex subcellular structures like mitochondria.

TABLE 7
IMT504 induced genes codifying extracellular
vesicle associated proteins

| Gene | Protein |
|---|---|
| TXN | Thioredoxin * |
| HSP90AB1 (#) | Heat Shock Protein 90 Alpha Family Class B |
| | Member 1 * |
| TRIP10 | Thyroid Hormone Receptor Interactor 10 |
| CD40 | CD40 Surface Antigen * |
| NCL | Nucleolin * |
| NCF2 | Neutrophil Cytosolic Factor 2 |
| PKM (#) | Pyruvate Kinase M1/2 * |
| ATIC | 5-Aminoimidazole-4-Carboxamide Ribonucleotide |
| | Formyltransferase/IMP Cyclohydrolase * |
| GRHPR | Glyoxylate and Hydroxypyruvate Reductase * |
| PRMT1 | Protein Arginine Methyltransferase 1 |
| TALDO1 | Transaldolase 1 * |
| FABP5 | Fatty Acid Binding Protein 5 |
| RAB13 | RAB13, Member RAS Oncogene Family |
| RANBP1 | RAN Binding Protein 1 |
| SCAMP3 | Secretory Carrier Membrane Protein 3 * |
| PSMB5 | Proteasome Subunit Beta 5 |
| MUC1 | Mucin 1, Cell Surface Associated |
| NME2 | NME/NM23 Nucleoside Diphosphate Kinase 2 * |
| PCBD | Pterin-4 Alpha-Carbinolamine Dehydratase |
| EIF5A | Eukaryotic Translation Initiation Factor 5A |
| GOT2 | Glutamic-Oxaloacetic Transaminase 2 * |
| EBNA1BP2 | EBNA1 Binding Protein 2 |
| DPP3 | Dipeptidyl Peptidase 3 * |
| CTSC | Cathepsin C |
| ENO1 (#) | Enolase 1 * |
| RUVBL2 | RuvB Like AAA ATPase 2 * |
| PSMD1 | Proteasome 26S Subunit, Non-ATPase 1 |
| IL-10 | Interleukin 10 |
| ALDOC | Aldolase, Fructose-Bisphosphate C * |
| NME1 | NME/NM23 Nucleoside Diphosphate Kinase 1 |
| NDUFC1 | NADH:Ubiquinone Oxidoreductase Subunit C1 |
| PSME2 | Proteasome Activator Subunit 2 * |
| ADSL | Adenylosuccinate Lyase |
| CSRP2 | Cysteine and Glycine Rich Protein 2 |
| IMPDH2 | Inosine Monophosphate Dehydrogenase 2 * |
| LGALS3 | Galectin 3 |
| TRAP1 | TNF Receptor Associated Protein 1 |
| FH | Fumarate Hydratase * |
| GNG10 | G Protein Subunit Gamma 10 * |
| PPIE | Peptidylprolyl Isomerase E |
| SNRPF | Small Nuclear Ribonucleoprotein Polypeptide F |
| CSK | C-Terminal Src Kinase |
| ACTG1 | Actin Gamma 1 |
| EIF2B3 | Eukaryotic Translation Initiation Factor 2B Subunit |
| | Gamma |
| LDHB (#) | Lactate Dehydrogenase B * |
| AKR1B1 | Aldo-Keto Reductase Family 1 Member B * |
| PRDX1 (#) | Peroxiredoxin 1 * |
| S100A4 | S100 Calcium Binding Protein A4 |
| ADA | Adenosine Deaminase |
| PSMD4 | Proteasome 26S Subunit, Non-ATPase 4 |
| BAX | BCL2 Associated X, Apoptosis Regulator * |
| MTHFD1 | Methylenetetrahydrofolate Dehydrogenase * |
| MDH2 | Malate Dehydrogenase 2 * |
| GBP1 | Guanylate Binding Protein 1 |
| EIF3C | Eukaryotic Translation Initiation Factor 3 Subunit C |
| SRM | Spermidine Synthase |
| CCL7 | C-C Motif Chemokine Ligand 7 |
| STAT5A | Signal Transducer And Activator Of Transcription 5A |
| RRAS2 | RAS Related 2 * |
| USP5 | Ubiquitin Specific Peptidase 5 |
| RPS26 | Ribosomal Protein S26 * |
| GSR | Glutathione-Disulfide Reductase |
| POLR2L | RNA Polymerase II Subunit L |
| FDPS | Farnesyl Diphosphate Synthase * |
| SOD1 | Superoxide Dismutase 1 |
| PFDN6 | Prefoldin Subunit 6 |
| EGF | Epidermal Growth Factor |
| VTI1B | Vesicle Transport Through Interaction With |
| | T-SNAREs 1B |
| ALDOA | Aldolase, Fructose-Bisphosphate A * |
| ACO1 | Aconitase 1 |
| PPP1R7 | Protein Phosphatase 1 Regulatory Subunit 7 |
| GPS1 | G Protein Pathway Suppressor 1 |
| AKR1A1 | Aldo-Keto Reductase Family 1 Member A1 * |
| TPI1 | Triosephosphate Isomerase 1 * |
| ETFA | Electron Transfer Flavoprotein Subunit Alpha |
| ACY1 | Aminoacylase 1 |
| NEDD8 | Neural Precursor Developmentally Down-Regulated |
| | 8 * |
| CBR1 | Carbonyl Reductase 1 |
| HSP90AA1 | Heat Shock Protein 90 Alpha Family Class A |
| | Member 1 * |
| PSME1 | Proteasome Activator Subunit 1 * |
| APRT | Adenine Phosphoribosyltransferase * |
| PA2G4 | Proliferation-Associated 2G4 * |
| POLR2H | RNA Polymerase II Subunit H |
| HSPB1 | Heat Shock Protein Family B (Small) Member 1 |
| SNRPD3 | Small Nuclear Ribonucleoprotein D3 Polypeptide |
| CFL1 | Cofilin 1 |
| ATP5J | ATP Synthase Peripheral Stalk Subunit F6 |
| PAFAH1B3 | Platelet Activating Factor Acetylhydrolase 1b Catalytic |
| | Subunit 3 |
| CD59 | CD59 Surface Antigen * |
| HLA-DR | Major Histocompatibility Complex, Class II, DR * |
| SSRP1 | Structure Specific Recognition Protein 1 |
| RAB34 | RAB34, Member RAS Oncogene Family |
| SNAP23 | Synaptosome Associated Protein 23 * |
| PPP1CA | Protein Phosphatase 1 Catalytic Subunit Alpha * |
| LSM2 | U6 Small Nuclear RNA and mRNA Degradation |
| | Associated |
| AP2S1 | Adaptor Related Protein Complex 2 Subunit Sigma 1 |
| PDCD5 | Programmed Cell Death 5 * |
| GALE | UDP-Galactose-4-Epimerase |
| GSTP1 | Glutathione S-Transferase Pi 1 |

All listed proteins in Table 7 have been described as components of exosomes excreted from non-tumor cells according to ExoCarta (exocarta.org/exosome_markers_new).
The symbol * means protein component of activated B cell exosomes as listed in ExoCarta.

### Example 3

### Examples of Early, Early-Late and Late Protein Synthesis by Purified Human CD19⁺B Cells Incubated with IMT504

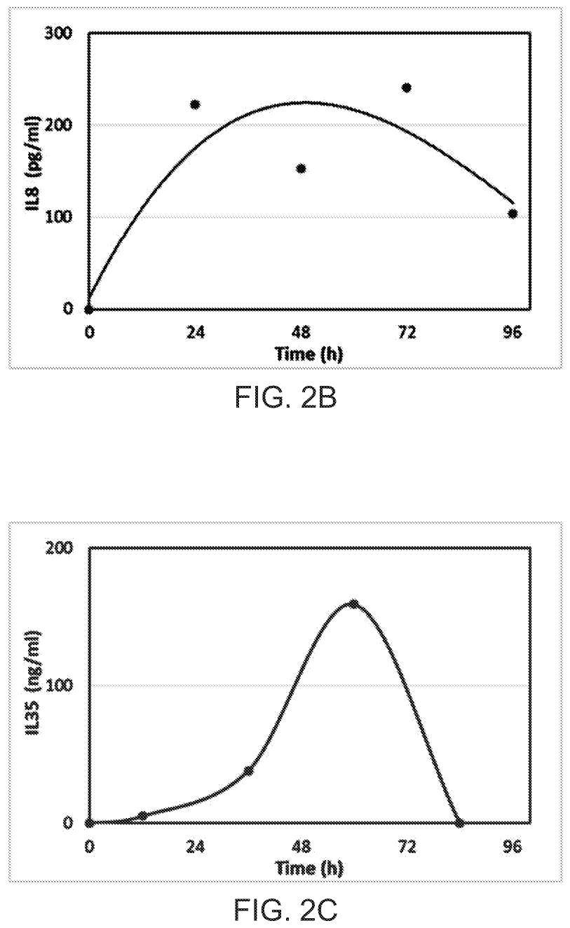

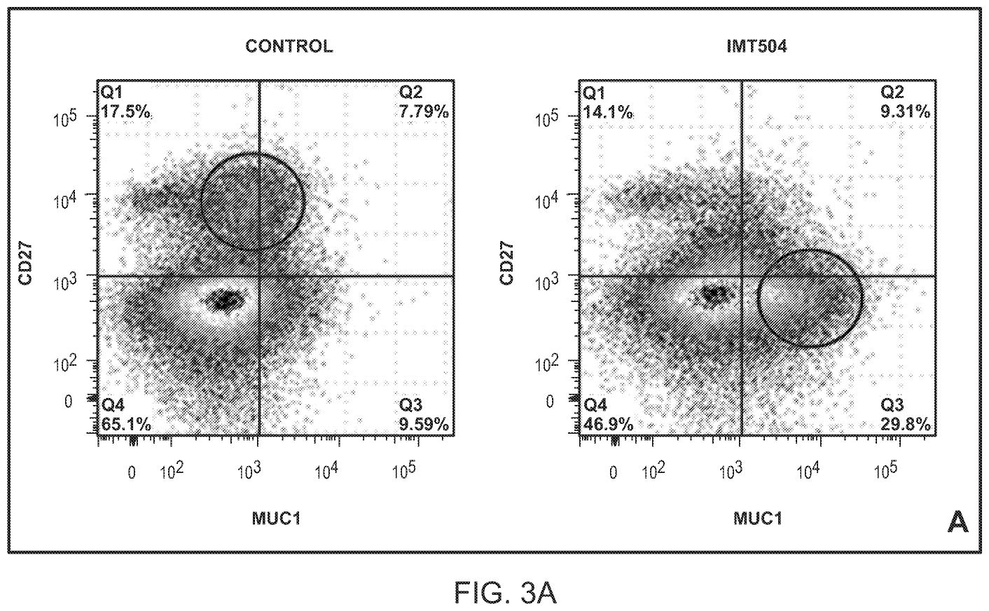

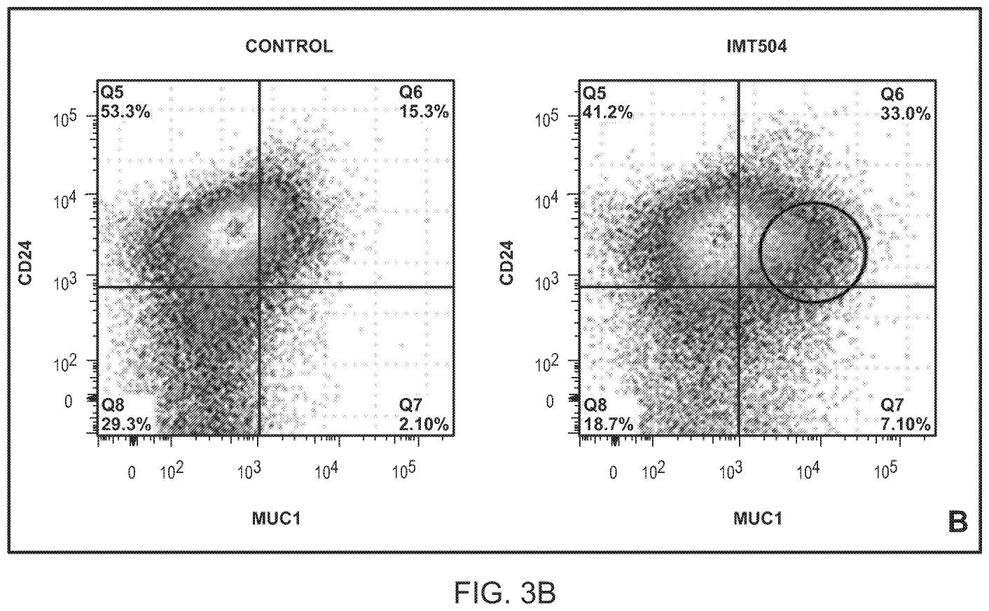

Selected gene products were investigated in order to validate the microarray results. FIG. 2A shows secretion of IL-10 (early induced gene product) by CD19⁺B cells incubated with IMT504 at different incubation times. Maximal secretion was observed at about 12 h of incubation with IMT504. FIG. 2B shows secretion of IL8 (early/late induced gene product). A stable secretion is reached at about 24 h of incubation with IMT504. FIG. 2C shows secretion of IL35, which include in its structure Ebi3, a late induced product according to microarray analysis. Maximal secretion was observed after about 60 h incubation with IMT504. It is noticeable that this late product is secreted in amounts about thousand times larger than early and early late gene products IL-10 and IL-8.

### Example 4

### Breg-Nov Cytological Analysts

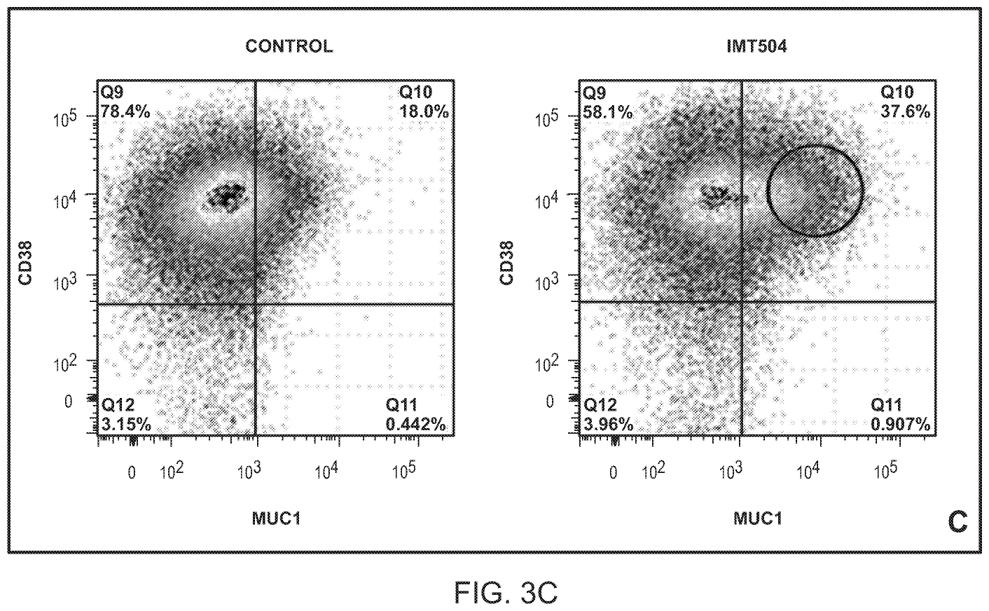

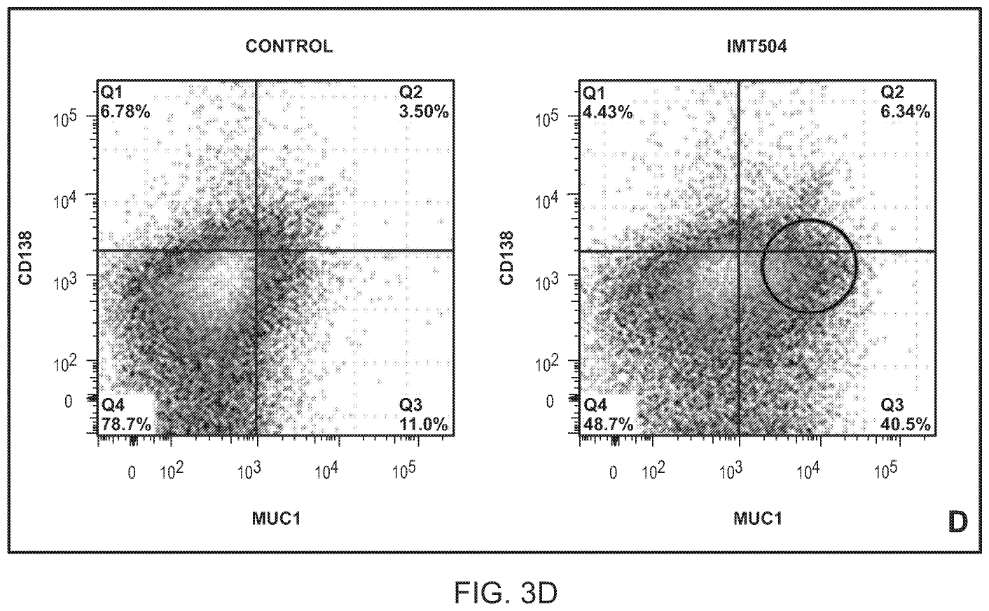

FIG. 3A shows that treatment of purified CD19⁺B cells with IMT504 results in a significant decrease of the CD27 marker corresponding to memory B cells and the upsurge of a population of cells with high expression of MUC1, a cell surface gene product corresponding to a late expressed gene according to the microarray analysis. Combination of MUC1 with the CD24 and the CD38 markers indicated that this strong expressing MUC1 population highly expresses both of these markers (FIG. 3B and FIG. 3C). In contrast, the highly expressing MUC1 population does not express the plasma cell CD138 marker (FIG. 3D). Results described in Examples 3 and 4 indicate that the Breg-nov described in this invention can be recognized as a subset of B cells Muc1^{high} CD24^{high} CD38^{high} CD27^{negative} CD138^{negative} which secrete large amounts of IL-35. This Breg-nov population is well established at about 48 h incubation of purified CD19⁺B cells with IMT504. However, after about 60 h incubation there is a marked loss in its capacity for IL-35 secretion, even though the cell viability is more than 85% after 72 h incubation as indicated in Example 1.

### Example 5

### Effect of Breg-Nov on the Myelination in the Brain of Demyelinated Rats

Experiments were conducted on a highly in-bred strain of Wistar rats raised in our own animal room and all procedures were in accordance with the NIH Guide for the Care and Use of Laboratory Animals. Experimental protocols were approved before implementation by the Institutional Committee for the Care and Use of Laboratory Animals (CICUAL) at Facultad de Farmacia y Bioquimica, Universidad de Buenos Aires, Argentina. Twenty-one-day-old rats of either sex were housed in a temperature- and photoperiod-controlled room and fed milled chow without (control) or 0.6% (w/w) cuprizone (CPZ) for 14 days until 35 days of age. Two days after CPZ withdrawal, animals were injected with saline solution (SS), IMT504 (20 mg/kg), 1×10⁵ SS-incubated lymphocyte B (48 h), or 1×10⁵ IMT504-incubated lymphocyte B according to the method of the invention. Animals were sacrificed 7 days after injection.

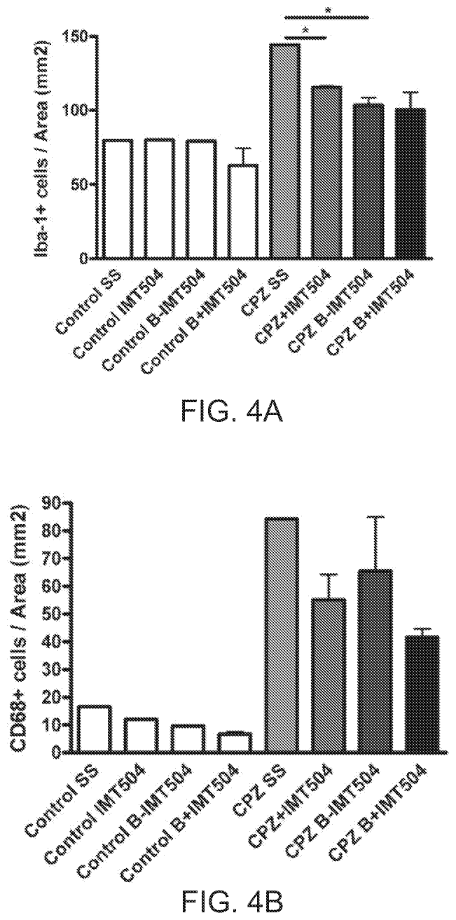

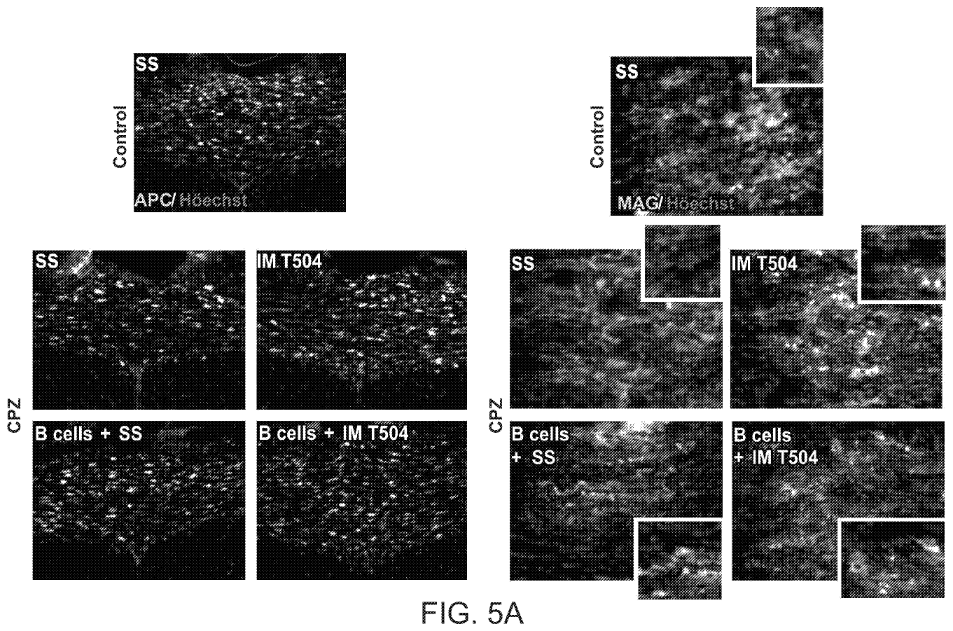

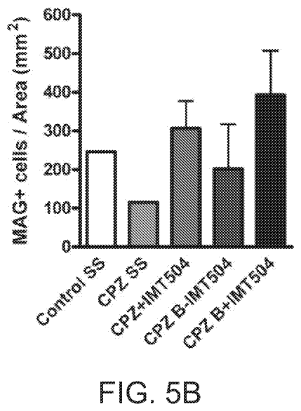

Briefly, 2 animals per experimental group were deeply anesthetized and perfused trans-intracardially with phosphate-buffered saline, pH 7.4 (PBS), followed by 4% (w/v) solution of paraformaldehyde in PBS. Brains were dissected out and post-fixed in the same solution overnight. After this, brains were thorough washed with PBS and cryoprotected by keeping them in 30% (w/v) sucrose in PBS. Brains were then frozen and used to obtain 30 μm free-floating coronal sections using a Leica CM 1850 cryotome. Microscopic observations were conducted using an Olympus BX50 microscope and photographs were obtained with a CoolSnap digital camera. The Image Pro Plus software (version 5.5) was used for image analysis. FIGS. 4A, 4B, 5A and 5B describe results. These results indicated that both IMT504 alone or IMT504-treated lymphocyte B cells induce an increase in the population of mature oligodendrocytes involved in the remyelination of the CC of CPZ-demyelinated rats. Results also showed a decrease in the inflammatory response observed as a consequence of demyelination in the CC.

### REFERENCES

1—Ray A, Dittel B N. Mechanisms of Regulatory B cell Function in Autoimmune and Inflammatory Diseases beyond IL-10. J Clin Med. 2017 Jan. 23; 6(1). pii: E12. doi: 10.3390/jcm6010012
2—Kawai T, Akira S, The role of pattern-recognition receptors in innate immunity: update on Toll-like receptors, Nat Immunol. 11 (2010) 373-384. doi:10.1038/ni.1863.
3—Zorzopulos J, Opal S M, Hernando-lnsúa A, Rodriguez J M, Elías F, Fló J, López R A, Chasseing N A, Lux-Lantos V A, Coronet M F, Franco R, Montaner A D, Horn D L. Immunomodulatory oligonucleotide IMT504: Effects on mesenchymal stem cells as a first-in-class immunoprotective/immunoregenerative therapy. World J Stem Cells. 2017; 9:45-67. doi: 10.4252/wjsc.v9i3.45.
4—Franco R, Rodriguez J M, Elías F, Hernando-Insúa A, Fló J, López R, Nagle C, Lago N, Zorzopulos J, Horn D L, Montaner A D. Non-Clinical Safety Studies of IMT504, a Unique Non-CpG Oligonucleotide. Nucleic Acid Ther. 2014; 24:267-282.
5—Hoffman W, Lakkis F G, Chalasani G. B Cells, Antibodies, and More. Clinical Journal of the American Society of Nephrology CJASN. 2016; 11(1):137-154. doi:10.2215/CJN.09430915.
6—Miyagaki T, Fujimoto M, Sato S. Regulatory B cells in human inflammatory and autoimmune diseases: from mouse models to clinical research. Int Immunol. 2015; 27:495-504. doi: 10.1093/intimm/dxv026.
7—Kimura, I., Konishi, M., Miyake, A., Fujimoto, M., and Itoh, N. (2006). Neudesin, a secreted factor, promotes neural cell proliferation and neuronal differentiation in mouse neural precursor cells. J. Neurosci, Res. 83, 1415-1424, doi: 10.1002/jnr.20849
8—Novais, A., Ferreira, A. C., Marques, F., Pêgo, J. M., Cerqueira, J. J., David-Pereira, A., et al. (2013). Neudesin is involved in anxiety behavior: structural and neurochemical correlates. Front. Behav. Neurosci. 7:119. doi: 10.3389/fnbeh.2013.00119
9—Toh H, Chitramuthu B P, Bennett H P, Bateman A. Structure, function, and mechanism of progranulin; the brain and beyond. J Mol Neurosci. 2011; 45:538-48. doi: 10.1007/s12031-011-9569-4
10—Yin F, Banerjee R. Thomas B et al (2010a) Exaggerated inflammation, impaired host defense, and neuropathology in progranulin deficient mice, J Exp Med. 18; 207:117-28. doi: 10.1084/jem.20091568
11—Jian, J., Tian., Q.-Y., Hettinghouse, A., Zhao, S., Liu, H., Wei, J., Liu, C. (2016), Progranulin Recruits HSP70 to β-Glucocerebrosidase and Is Therapeutic Against Gaucher Disease. EBioMedicine, 13, 212-224. doi.org/10.1016/j.ebiom.2016.10.010
12—Yu, Y., Xu, X., Liu, L., Mao, S., Feng, T., Lu, Y.,Tang, W. (2016). Progranulin deficiency leads to severe inflammation, lung injury and cell death in a mouse model of endotoxic shock. Journal of Cellular and Molecular Medicine, 20(3), 506-517. doi. org/10.1111/jcmm.12756
13—Huang, K., Chen, A., Zhang, X., Song, Z., Xu, H., Cao, J., & Yin, Y. (2015). Progranulin is preferentially expressed in patients with psoriasis vulgaris and protects mice from psoriasis-like skin inflammation. Immunology, 145, 279-287. doi.org/10.1111/imm.12446
14—Díaz-Alvarez, L., & Ortega, E. (2017). The Many Roles of Galectin-3, a Multifaceted Molecule, in Innate Immune Responses against Pathogens. Mediators of Inflammation, 2017, 9247574. doi.org/10.1155/2017/9247574
15—Souza, B. S. de F., da Silva, K. N., Silva, D. N., Rocha, V. P. C., Paredes, B. D., Azevedo, C. M., . . . Soares, M. B. P. (2017). Galectin-3 Knockdown Impairs Survival, Migration, and Immunomodulatory Actions of Mesenchymal Stromal Cells in a Mouse Model of Chagas Disease Cardiomyopathy. Stem Cells International, 2017, 3282656. doi.org/10.1155/2017/3282656
16—Hsieh W C, Mackinnon A C, Lu W Y, Jung J, Boulter L, Henderson N C, Simpson K J, . . . Forbes S J1. Galectin-3 regulates hepatic progenitor cell expansion during liver injury. Gut. 2015; 64:312-21. doi: 10.1136/gutjnl-2013-306290
17—Gao, Q., Xia, Y., Liu, L., Huang, L., Liu, Y., Zhang, X., . . . Li, K. (2016). Galectin-3 Enhances Migration of Miniature Pig Bone Marrow Mesenchymal Stem Cells Through Inhibition of RhoA-GTP Activity. Scientific Reports, 6, 26577. doi.org/10.1038/srep26577
18—Zeng, F., & Harris, R. C. (2014). Epidermal growth factor, from gene organization to bedside. Seminars in Cell & Developmental Biology, 0, 2-11. doi.org/10.1016/j.semcdb.2014.01.011
19—Suzuki, Y., Yanagisawa, M., Yagi, H., Nakatani, Y., & Yu, R. K. (2010). Involvement of β1-Integrin Up-regulation in Basic Fibroblast Growth Factor- and Epidermal Growth Factor-induced Proliferation of Mouse Neuroepithelial Cells. The Journal of Biological Chemistry, 285, 18443-18451. doi.org/10.1074/jbc.M110.114645
20—Garcez R C, Teixeira B L, Schmitt Sdos S, Alvarez-Silva M, Trentin A G. Epidermal growth factor (EGF) promotes the in vitro differentiation of neural crest cells to neurons and melanocytes. Cell Mol Neurobiol. 2009; 29:1087-91, doi: 10.1007/s10571-009-9406-2
21—Aghila Rani K G, Kartha C C. Effects of epidermal growth factor on proliferation and migration of cardiosphere-derived cells expanded from adult human heart. Growth Factors. 2010; 28:157-165.doi: 10.3109/08977190903512628
22—Tamama K., Kawasaki, H., & Wells, A. (2010). Epidermal Growth Factor (EGF) Treatment on Multipotential Stromal Cells (MSCs). Possible Enhancement of Therapeutic Potential of MSC. Journal of Biomedicine and Biotechnology, 2010, 795385. doi.org/10.1155/2010/795385
23—Suzuki A, Sekiya S, Gunshima E, Fujii S, Taniguchi H. EGF signaling activates proliferation and blocks apoptosis of mouse and human intestinal stem/progenitor cells in long-term monolayer cell culture. Laboratory investigation; a journal of technical methods and pathology. 2010; 90:1425-1436.doi: 10.1038/labinvest.2010.150
24—Nanba D, Toki F, Barrandon Y, Higashiyama S. Recent advances in the epidermal growth factor receptor/ligand system biology on skin homeostasis and keratinocyte stern cell regulation. J Dermatol Sci. 2013; 72:81-86.doi: 10.1016/j.jdermsci.2013.05.009
25—Mohammed, M. K., Shao, C., Wang, J., Wei, Q., Wang, X., Collier, Z., Lee, M. J. (2016). Wnt/β-catenin signaling plays an ever-expanding role in stem cell self-renewal, tumorigenesis and cancer chemoresistance. Genes & Diseases, 3, 11-40. doi.org/10.1016/j.gendis.2015.12.004
26—Bengoa-Vergniory N, Kypta R M. Canonical and noncanonical Wnt signaling in neural stem/progenitor cells. Cell Mol Life Sci. 2015; 72:4157-72. doi: 10.1007/s00018-015-2028-6
27—Kretzschmar K, Clevers H. Wnt/β-catenin signaling in adult mammalian epithelial stern cells. Dev Biol. 2017 Aug. 15; 428:273-282. doi: 10.1016/j.ydbio.2017.05.015
28—Wei Q, Zhang J, Hong G, Chen Z, Deng W, He W, Chen MH. Icariin promotes osteogenic differentiation of rat bone marrow stromal cells by activating the ERα-Wnt/β-catenin signaling pathway. Biomed Pharmacother. 2016; 84: 931-939. doi: 10.016/j.biopha.2016.09.107
29—Clevers H, Loh K M, Nusse R. Stem cell signaling. An integral program for tissue renewal and regeneration: Wnt signaling and stem cell control. Science. 2014; 346(6205):1248012. doi: 10.1126/science.1248012
30—Hasegawa, H., Mizoguchi, I., Chiba, Y., Ohashi, M., Xu, M., & Yoshimoto, T. (2016). Expanding Diversity in Molecular Structures and Functions of the IL-6/IL-12 Heterodimeric Cytokine Family. Frontiers in Immunology, 7, 479. doi.org/10.3389/fimmu.2016.00479
31—Egwuagu, C. E., & Yu, C.-R. (2015). Interleukin 35—Producing B Cells (i35-Breg): A New Mediator of Regulatory B-Cell Functions in CNS Autoimmune Diseases. Critical Reviews in Immunology, 35, 49-57
32—Shen, P., Roch, T., Lampropoulou, V., O'Connor, R A., Stervbo, U., Hilgenberg, E., Fillatreau, S. (2014). IL-35-producing B cells are critical regulators of immunity during autoimmune and infectious diseases. Nature, 507(7492), 366-370. doi.org/10.1038/nature12979
33—Liu, Y., Wu, Y., Wang, Y., Cai, Y., Hu, B., Bao, G., . . . Liu, H. (2015). IL-35 mitigates murine acute graft-versus-host disease with retention of graft-versus-leukemia effects. Leukemia, 29, 939-946, doi.org/10.1038/leu.2014.310
34—Zhang X H, Zhou Y, Zhang J M, Zhou S Y, Wang M, Feng R, Feng F E, Wang Q M, Zhu X L, Zhao X S, Lv M, Kong Y, Chang Y J, Huang X J. IL-35 inhibits acute graft-versus-host disease in a mouse model. Int Immunopharmacol. 2015; 29:383-392. doi: 10.1016/j.intimp.2015.10.025
35—Cai, Z., Wong, C. K., Dong, J., Chu, M., Jiao, D., Kam, N. W., . . . Tam, L. S. (2015). Remission of systemic lupus erythematosus disease activity with regulatory cytokine interleukin (IL)-35 in Murphy Roths Large (MRL)/lpr mice. Clinical and Experimental Immunology, 181, 253-266. doi.org/10.1111/cei.12639
36—Manzoor F, Johnson M C, Li C, Samulski R J, Wang B, Tisch R. β-cell-specific IL-35 therapy suppresses ongoing autoimmune diabetes in NOD mice. Eur J Immunol. 2017; 47:144-154. doi: 10.1002/eji.201646493
37—Park, M. S., Choi, S., Lee, Y. R., Joo, H. K., Kang, G. Kim, C.-S., . . . Jeon, B. H. (2016). Secreted APE1/Ref-1 inhibits TNF-α-stimulated endothelial inflammation via thiol-disulfide exchange in TNF receptor. Scientific Reports, 6, 23015. doi.org/10.1038/srep23015
38—Baek, H., Lim, C. S., Byun, H. S., Cho, H. S., Lee, Y. R., Shin, Y. S., . . . Park, J. B. (2016). The anti-inflammatory role of extranuclear apurinic/apyrimidinic endonuclease 1/redox effector factor-1 in reactive astrocytes. Molecular Brain, 9, 99. doi.org/10.1186/s13041-016-0280-9
39—Leak, R. K., Li, P., Zhang, F., Sulaiman, H. H., Weng, Z., Wang, G., . . . Chen, J. (2015). Apurinic/Apyrimidinic Endonuclease 1 Upregulation Reduces Oxidative DNA Damage and Protects Hippocampal Neurons from Ischemic Injury. Antioxidants & Redox Signaling, 22, 135-148. doi.org/10.1089/ars.2013.5511
40—Yu, Y., Cui, Y., Zhao, Y., Liu, S., Song, G., Jiao, P., . . . Qin, S. (2016). The binding capability of plasma phospholipid transfer protein, but not HDL pool size, is critical to repress LPS induced inflammation. Scientific Reports, 6, 20845. doi.org/10.1038/srep20845
41—Deckert, V., Lemaire, S., Ripoll, P.-J., de Barros, J.-P. P., Labbé, J., Borgne, C. C.-L., . . . Lagrost, L. (2017), Recombinant human plasma phospholipid transfer protein (PLTP) to prevent bacterial growth and to treat sepsis. Scientific Reports, 7, 3053. doi.org/10.1038/s41598-017-03285-9
42—Ueno, K., Koga, T., Kato, K., Golenbock, D. T., Gendler, S. J., Kai, H., & Kim, K. C. (2008). MUC1 Mucin Is a Negative Regulator of Toll-Like Receptor Signaling. American Journal of Respiratory Cell and Molecular Biology, 38, 263-268. doi.org/10.1165/rcmb.2007-0336RC
43—Ng G Z, Sutton P. The MUC1 mucin specifically inhibits activation of the NLRP3 inflammasome. Genes Immun. 2016 April; 17:203-6. doi: 10.1038/gene.2016.10
44—Kalinski P. (2012). Regulation of immune responses by prostaglandin E2. J Immunol. 188:21-28. doi: 10.4049/jimmunol.1101029
45—Mittal, M., Siddiqui, M. R., Tran, K., Reddy, S. P., & Malik, A. B. (2014). Reactive Oxygen Species in Inflammation and Tissue Injury. Antioxidants & Redox Signaling, 20, 1126-1167. doi.org/10.1089/ars.2012.5149
46—Teixeira J H, Silva A M, Almeida M I, Barbosa M A, Santos S G. (2016). Circulating extracellular vesicles: Their role in tissue repair and regeneration. Transfus Apher Sci, 55:53-61. doi: 10.101.6/j.transci.2016.07.015
47—Robbins P D, Dorronsoro A, Booker C N. (2016). Regulation of chronic inflammatory and immune processes by extracellular vesicles. J Clin Invest. 126:1173-80. doi: 10.1172/JCI81131
48—Paliwal S, Chaudhuri R, Agrawal A, Mohanty S. (2018). Regenerative abilities of mesenchymal stem cells through mitochondrial transfer. J Biomed Sci, 25:31. doi: 10.1186/s12929-018-0429-1
49—Dianzani C, Bellavista E, Liepe J, Verderio C, Mishto M. (2017). Extracellular proteasome-osteopontin circuit regulates cell migration with implications in multiple sclerosis. Sci Rep. 2017 Mar. 9; 7:43718. doi: 10.1038/srep43718
50—Riley, J. L., June, C. H., & Blazar, B. R. (2009). Human T Regulatory Cells as Therapeutic Agents: Take a Billion or So of These and Call Me in the Morning. Immunity, 30, 656-665. doi.org/10.1016/j.immuni.2009.04.006
51—Robbins, P. D., & Morelli, A. E. (2014). Regulation of immune responses by extracellular vesicles. Nature reviews. Immunology, 14, 195-208. doi:10.1038/nri3622
52—Paliwal, S., Chaudhuri, R., Agrawal, A., & Mohanty. S. (2018). Regenerative abilities of mesenchymal stem cells through mitochondrial transfer. Journal of biomedical science, 25, 31-43. doi:10.1186/s12929-018-0429-1
53—Teixeira J H, Silva A M, Almeida M, Barbosa M A, Santos S G (2016). Circulating extracellular vesicles: Their role in tissue repair and regeneration. Transfus Apher Sci, 55:53-61. doi: 10.1016/j.transci.2016.07.015
54—Pinheiro A, Silva A M, Teixeira J H, Gonçalves R M, Almeida M I, Barbosa M A, Santos S G. (2018). Extracellular vesicles: intelligent delivery strategies for therapeutic applications. J Control Release. 24; 289:56-69. doi: 10.1016/j.jconrel.2018.09.019
55—Raposo G, Stoorvogel W. (2013). Extracellular vesicles: exosomes, microvesicles, and friends. The Journal of cell biology, 200: 373-383. doi: 10.1083/jcb.201211138
56—Pylayeva-Gupta Y. (2016). Molecular Pathways: Interleukin-35 in Autoimmunity and Cancer. Clin Cancer Res, 15: 4973-4978; doi: 10.1158/1078-0432.CCR-16-0743
57—Niedbala W, Wei X Q, Cai B, Hueber A J, Leung B P, McInnes I B, Liew F Y. (2007). IL-35 is a novel cytokine with therapeutic effects against collagen-induced arthritis through the expansion of regulatory T cells and suppression of Th17 cells. Eur J Immunol., 237:3021-9. doi: 10.1002/eji.200737810
58—Crampton, S. P., Voynova, E., & Bolland, S. (2010). Innate Pathways to B cell Activation and Tolerance. Annals of the New York Academy of Sciences, 1183, 58-68. doi.org/10.1111/j.1749-6632.2009.05123.x
59—Lund, F. E. (2008). Cytokine-producing B lymphocytes—key regulators of immunity. Current Opinion in Immunology, 20, 332-338, doi.org/10.1016/j.coi.2008.03.003
60—Baratelli F, Lin Y, Zhu L, Yang S C, Heuzé-Vourc'h N, Zeng G, Reckamp K, Dohadwala M, Sharma S, Dubinett S M. (2005). Prostaglandin E2 induces FOXP3 gene expression and T regulatory cell function in human CD4⁺ T cells. J Immunol. 175:1483-90. doi: 10.4049/jimmunol.175.3.1483 8.03.003

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed is:

1. A method of producing a regulatory B cell comprising: contacting one or more B cells, with a phosphorothioate oligonucleotide having at least a 50% sequence identity to SEQ ID NO: 1, wherein the B cells are CD19⁺ cells.

2. The method of claim 1, wherein the phosphorothioate oligonucleotide has a sequence of at least 70% to SEQ ID NO: 1.

3. The method of claim 1, wherein the CD19⁺ B cells produce one or more proteins associated with immune regulatory and/or tissue reparatory processes.

4. The method of claim 3, wherein the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 or Prostaglandin E2 synthase.

5. The method of claim 1, wherein the B cells are contacted with the phosphorothioate oligonucleotide for at least about 30 minutes.

6. A method of suppressing an autoimmune response in a subject, comprising obtaining B cells; culturing and contacting the B cells with a phosphorothioate oligonucleotide having at least a 50% sequence identity to SEQ ID NO: 1, administering the B cells to the subject, thereby suppressing the autoimmune response in the subject.

7. The method of claim 6, wherein the phosphorothioate oligonucleotide has a sequence of at least 70% to SEQ ID NO: 1.

8. The method of claim 6, wherein the CD19⁺ B cells produce one or more proteins associated with immune regulatory and/or tissue reparatory processes.

9. The method of claim 8, wherein the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 or Prostaglandin E2 synthase.

10. The method of claim 6, wherein the B cells are cultured ex vivo.

11. The method of claim 6, wherein the B cells are autologous, haplotype matched, cell-lines, stem cells or combinations thereof.

12. The method of claim 6, further comprising administering one or more immunosuppressive agents and/or a phosphorothioate oligonucleotide having at least a 50% sequence identity to SEQ ID NO: 1.

13. A method of suppressing acute or chronic inflammation or repairing a damaged organ or tissue in mammals comprising obtaining B cells; culturing and contacting the B cells with a phosphorothioate oligonucleotide having at least a 50% sequence identity to SEQ ID NO: 1, administering the B cells to the subject, thereby suppressing the autoimmune response in the subject.

14. The method of claim 13, wherein the phosphorothioate oligonucleotide has a sequence of at least 70% to SEQ ID NO: 1.

15. The method of claim 13, wherein the CD19⁺ B cells produce one or more proteins associated with immune regulatory and/or tissue reparatory processes.

16. The method of claim 15, wherein the one or more proteins associated with immune regulatory and/or tissue reparatory processes, comprise: neudesin, pro-Granulin, Galectin 3, Epidermal Growth Factor, Wnt Family Member 8B, Secreted Frizzled Related Protein 5, Epstein-Barr Virus Induced 3, Apurinic/Apyrimidinic Endonuclease 1, Phospholipid transfer protein, Mucin 1, Integrin Subunit Alpha 2 or Prostaglandin E2 synthase.

17. The method of claim 13, wherein the B cells are cultured ex vivo.

18. The method of claim 13, wherein the B cells are autologous, haplotype matched, cell-lines, stem cells or combinations thereof.

19. The method of claim 13, further comprising administering one or more anti-inflammatory agents, other therapeutics and/or a phosphorothioate oligonucleotide having at least a 50% sequence identity to SEQ ID NO: 1.

20. A composition comprising a therapeutically effective amount of a phosphorothioate oligonucleotide having at least a 50% sequence identity to SEQ ID NO: 1 (IMT504), one or more anti-inflammatory agents, other therapeutics, immunosuppressive agents, chemotherapeutic agents or combinations thereof.

21. A method of treating cancer comprising: comprising obtaining B cells; culturing and contacting the B cells with a phosphorothioate oligonucleotide having at least a 50% sequence identity to SEQ ID NO: 1, administering the B cells to the subject, thereby treating cancer.

22. The method of claim 21, further comprising administering one or more chemotherapeutic agents and/or a phosphorothioate oligonucleotide having at least a 50% sequence identity to SEQ ID NO: 1.

23. A method of regulating an immune response, comprising: contacting one or more cells, with a phosphorothioate oligonucleotide having at least a 50% sequence identity to SEQ ID NO: 1.

24. The method of claim 23, wherein the phosphorothioate oligonucleotide has a sequence of at least 70% to SEQ ID NO: 1.

25. The method of claim 23, wherein the one or more cells comprise immune cells.

26. The method of claim 25, wherein the immune cells comprise: B cells, T cells, antigen presenting cells, chimeric antigen receptor-T cells (CAR-T) or combinations thereof.

27. The method of claim 23, wherein the cells are autologous cells, comprising: autologous, allogeneic, haplotype matched, haplotype mismatched, haplo-identical, xenogeneic, cell lines or combinations thereof.

28. A method of treating cancer comprising: comprising obtaining immune cells; culturing and contacting the immune cells with a composition comprising a phosphorothioate oligonucleotide having at least a 50% sequence identity to SEQ ID NO: 1 and/or a tumor antigen, and administering the immune cells to the subject, thereby treating cancer.

29. The method of claim 28, further comprising administering one or more chemotherapeutic agents and/or a phosphorothioate oligonucleotide having at least a 50% sequence identity to SEQ ID NO: 1.

30. The method of claim 28, wherein the immune cells comprise: B cells, T cells, antigen presenting cells, chimeric antigen receptor-T cells (CAR-T) or combinations thereof.

31. The method of claim 30, wherein the cells are autologous cells, comprising: autologous, allogeneic, haplotype matched, haplotype mismatched, haplo-identical, xenogeneic, cell lines or combinations thereof.

32. A method of producing regulatory B (Breg) cells, comprising contacting B cells ex vivo with a single stranded immunomodulatory oligonucleotide IMT504 with the TCATCATTTTGTCATTTTGTCATT (SEQ ID NO: 1) sequence for about 48 hours wherein the Breg cells produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase.

33. A method for preparing immunomodulatory extracellular vesicles, comprising contacting B-cells ex vivo with an immunomodulatory oligonucleotide having a sequence as set forth in SEQ ID NO: 1 for about 48 hours, and recovering the extracellular vesicles from the cell culture supernatant.

34. A method of producing the cytokine interleukin-35 (IL-35), comprising contacting B cells ex vivo with an immunomodulatory oligonucleotide comprising SEQ ID NO: 1 (IMT504) for about 48 hours, and recovering the IL-35 from the cell culture supernatant.

35. The method of any one of claims 32-34, were the B cells are primary B cells.

36. The method of any one of claims 32-34, were the B cells are cell-line B cells.

37. A method for differentiating cells to an anti-inflammatory and/or pro-reparatory tissue/organ stage or for proliferating cells, in vitro or in vivo, comprising contacting the B cells obtained according to claim 32 with the cells.

38. The method of claim 37, wherein the cells are monocytes.

39. The method of claim 37, wherein the cells are T cells

40. The method of claim 37, wherein the cells are stem cells.

41. A method for autoimmunity treatment in a mammal, comprising administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase prepared according to the method of claim 32.

42. A method for inflammatory disease treatment in a mammal, comprising administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase wherein the B cells are prepared according to the method of claim 32.

43. The method of claim 42, wherein the inflammatory disease is a chronic inflammatory disease.

44. A method for graft versus host disease prevention or treatment in a mammal, comprising administering to the mammal B cells that produce Neudesin, Pro-Granulin, Galectin 3, Epidermal Growth Factor, Wnt Family Member 8B, Interleukin 35, Apurinic/Apyrimidinic Endonuclease 1, Mucin 1 and Prostaglandin E2 synthase prepared according to the method of claim 32.

45. A method for autoimmunity treatment in a mammal, which comprises administering to the mammal extracellular vesicles prepared by the method of claim 33.

46. A method for inflammatory disease treatment in a mammal, which comprises administering to the mammal exosomes prepared by the method of claim 33.

47. The method of claim 46, wherein the inflammatory disease is a chronic inflammatory disease.

48. A method for graft versus host disease prevention or treatment in a mammal, which comprises administering to the mammal extracellular vesicles prepared by the method of claim 33.
